(19) United States
(12) Patent Application Publication
(10) Pub. No.: US 2003/0159177 A1
(43) Pub. Date: Aug. 21, 2003

(54) Polynucleotides encoding polypeptides having invertase activity and use of same

(76) Inventors: Danny Zamir, Gedera (IL); Pleban Tzili, Kiryat Ono (IL); Fridman Eyal, Rehovot (IL)

Correspondence Address: SOL SHEINBEIN, G.E. EHRLICH (1995) LTD., c/o ANTHONY CASTORINA, 2001 JEFFERSON DAVIS HIGHWAY, SUITE 207, ARLINGTON, VA, 22202, US

(21) Appl. No.: 10/070,923

(22) Filed: Mar. 13, 2002

Related U.S. Application Data
(63) Continuation-in-part of application No. PCT/IL01/00008, filed on Jan. 2, 2001.
(63) Continuation-in-part of application No. 09/477,375, filed on Jan. 4, 2000, now abandoned.

(51) Int. Cl. A01H 1/00; C12Q 1/68; C12N 9/90; C12N 15/82; C12N 5/04

(52) U.S. Cl. 800/284; 435/6; 435/69.1; 435/320.1; 435/419; 435/233

(57) ABSTRACT

An isolated nucleic acid comprising a genomic, complementary or composite polynucleotide sequence encoding a polypeptide having an invertase activity in an apoplastic environment and an N terminal amino acid sequence serving for secretion into an apoplast.

This application is a Continuation-In-Part of PCT/IL01/00008, filed Jan. 2, 2001, and U.S. patent application Ser. No. 09/477,375, filed Jan. 4, 2000.

### FIELD AND BACKGROUND OF THE INVENTION

The present invention relates to isolated polynucleotides encoding polypeptides having invertase activity, constructs including same and methods of utilizing same. More particularly, the present invention relates to isolated polynucleotides encoding novel invertases, which polynucleotides can be used for substantially increasing the sugar content in for example, fruits, roots, leaves, etc., of plants expressing same. In addition, the present invention relates to a novel regulatory sequence which when integrated, in a site specific manner, into a solanaceae plant genome, can substantially increase the sugar content in tissues, such as for example, fruits, etc., of the solanaceae plant.

Ever since the emergence of modern agriculture, agricultured plants have been manipulated in an effort to establish crops with agronomically important traits.

Such traits typically include, plant yield and quality, enhanced growth rates and adaptation to various growth conditions.

At presents a great deal of emphasis is placed on the generation of plants having desired traits via genetic engineering techniques. However, since these traits are often the result of the activity of several genes (referred to as quantitative traits), the use of direct gene transfer in manipulating these traits, is difficult due to problems in pinpointing and then cloning the individual loci which contribute predominantly to the expression of the trait.

As such, genetic manipulation of plants is typically practiced using conventional breeding techniques, such as hybrid crossing.

Although utilizing such breeding techniques typically enables breeders to interogress quantitative trait loci of a specific function into a desirable genetic background, such conventional breeding techniques suffer from several limitations.

Oftentimes the “isolation” of a single trait loci (referred to as quantitative trait loci or QTL) can be difficult due to linkage drag, or due to effects of epistatic QTLs present in the genetic background or in chromosomal association with the single trait loci.

One example of a plant which has been extensively bred is tomato.

A major objective in tomato breeding is to increase the content of total soluble solids (TSS or brix; mainly sugars and acids) of the fruits in order to improve taste and processing qualities.

As such, efforts have been made to interogress the high fruit sugar content of wild Lycopersicon species which is three times higher than cultivated varieties into cultivated varieties which are characterized by a large fruit mass, small foliage, concentrated ripening and other commercially desirable traits.

To try and resolve the genetic basis for-the high sugar content of fruits of wild Lycopersicon species, Eshed and Zamir (Genetics 141: 1147-1162, 1995; Genetics 143: 1807-1817, 1996, both are incorporated herein by reference) developed a set of 50 introgression lines from a cross between the green-fruited species L. pennelli and the cultivated tomato, L. esctulentum.

Each of the lines contained a single RFLP defined L. pennelli chromosome segment, and together the lines provide complete coverage of the tomato genome. Using this resource it was possible to map 23 QTLs that regulate brix.

Although this research work presents significant progress in determining the QTLs responsible for a high brix value, plants generated by introgressing L. pennelli into the cultivated tomato, L. esculentum genetic background are of little commercial value since their phenotype, in many aspects, is closer to that of the wild Lycopersicon species.

In order to generate hybrids characterized by a uniform ripening, a good cover of the fruit and a high brix value, which hybrids are of high commercial value, it is necessary to narrow the introgression described by Eshed and Zamir in order to isolate the brix QTL.

Furthermore, since genetic crossing is genus limited, in order to enable generation of a high brix value in plants unbreedable with tomato plants, a gene or genes responsible for the high brix value in L. pennelli must be isolated, which gene or genes when introduced and expressed in a plant other than tomato substantially increase the fruit brix value thereof.

Thus, the present invention describes the isolation of polynucleotides which encode for novel plant invertases which are associated with the high brix value in L. pennelli fruit. The present invention further describes recombinant methods which utilize these isolated polynucleotides for increasing the brix value of plant tissues.

### SUMMARY OF THE INVENTION

According to one aspect of the present invention there is provided an isolated nucleic acid comprising a genomic, complementary or composite polynucleotide sequence encoding a polypeptide having an invertase activity in an apoplastic environment and an N terminal amino acid sequence serving for secretion into an apoplast.

According to further features in preferred embodiments of the invention described below, the polypeptide is at least 80% homologous to SEQ ID NO:0.5, as determined using the BestFit software of the Wisconsin sequence analysis package, utilizing the Smith and Waterman algorithm, where gap creation penalty equals 8 and gap extension penalty equals 2.

According to still further features in the described preferred embodiments the polynucleotide is at least 80% identical with SEQ ID NO:6 as determined using the BestFit software of the Wisconsin sequence analysis package, utilizing the Smith and Waterman algorithm, where gap weight equals 50, length weight equals 3, average match equals 10 and average mismatch equals −9.

According to another aspect of the present invention there is provided an isolated nucleic acid comprising a genomic, complementary or composite polynucleotide sequence encoding a polypeptide having an invertase activity, the polypeptide is at least 80% homologous to SEQ ID NO:5, as determined using the BestFit software of the Wisconsin sequence analysis package, utilizing the Smith and Waterman algorithm, where gap creation penalty equals 8 and gap extension penalty equals 2.

According to further features in preferred embodiments of the invention described below, the polynucleotide is hybridizable with SEQ ID NOs:1, 4 or 6 under hybridization conditions of hybridization solution containing 10% dextrane sulfate, 1 M NaCl, 1% SDS and 5×10⁶ cpm ³²p labeled probe, at 65° C., with a final wash solution of 0.1×SSC and 0.1% SDS and final wash at 60° C.

According to still further features in the described preferred embodiments the polynucleotide is at least 80% identical with SEQ ID NO:6 as determined using the BestFit software of the Wisconsin sequence analysis package, utilizing the Smith and Waterman algorithm, where gap weight equals 50, length weight equals 3, average match equals 10 and average mismatch equals −9.

According to still further features in the described preferred embodiments the polypeptide is as set forth in SEQ ID NO:5 or portions thereof.

According to still further features in the described preferred embodiments thc polynucleotide is as set forth in SEQ ID NO:6 or portions thereof.

According to yet another aspect of the present invention there is provided a nucleic acid construct comprising any of the isolated nucleic acids described hereinabove.

According to still further features in the described preferred embodiments the nucleic acid construct further comprising a promoter for regulating expression of the isolated nucleic acid in an orientation selected from the group consisting of sense and antisense orientation.

According to still further features in the described preferred embodiments the nucleic acid construct further comprising a positive and a negative selection markers for selecting for homologous recombination events.

According to still another aspect of the present invention there is provided a plant cell, tissue or a whole plant comprising any of the nucleic acid constructs described herein.

According to an additional aspect of the present invention there is provided a recombinant protein comprising a polypeptide having an invertase activity in an apoplastic environment and an N terminal amino acid sequence serving for secretion into an apoplast.

According to still farther features in the described preferred embodiments the polypeptide is at least 80% homologous to SEQ ID NO:5, as determined using the BestFit software of the Wisconsin sequence analysis package, utilizing the Smith and Waterman algorithm, where gap creation penalty equals 8 and gap extension penalty equals 2.

According to still further features in the described preferred embodiments the polypeptide includes at least a portion of SEQ ID NO:5.

According to still further features in the described preferred embodiments the protein is encoded by a polynucleotide hybridizable with SEQ ID NOs:1, 4 or 6 or a portion thereof under hybridization conditions of hybridization solution containing 10% dextrane sulfate, 1 M NaCl, 1% SDS and 5×10⁶ cpm ³²p labeled probe, at 65° C., with a final wash solution of 0.1×SSC and 0.1% SDS and final wash at 60° C.

According to still further features in the described preferred embodiments the protein is encoded by a polynucleotide at least 80% identical with SEQ ID NO:6 or portions thereof as determined using the BestFit software of the Wisconsin sequence analysis package, utilizing the Smith and Waterman algorithm, where gap weight equals 50, length weight equals 3, average match equals 10 and average mismatch equals −9.

According to still further features in the described preferred embodiments the recombinant protein comprising a polypeptide as set forth in SEQ ID NO:5.

According to yet an additional aspect of the present invention there is provided a method of increasing a level of a monosaccharide in a plant tissue, the method comprising the step of expressing in the plant tissue a polypeptide having invertase activity, wherein the polypeptide is at least 80% homologous to SEQ ID NO:5 as determined using the BestFit software of the Wisconsin sequence analysis package, utilizing the Smith and Waterman algorithm, where gap creation penalty equals 8 and gap extension penalty equals 2.

According to still an additional aspect of the present invention there is provided a method of increasing a level of a monosaccharide in a plant tissue, the method comprising the step of expressing a polypeptide having invertase activity, wherein the polypeptide is encoded by a polynucleotide hybridizable with SEQ ID NOs: 1, 4 or 6 or a portion thereof under hybridization conditions of hybridization solution containing 10% dextrane sulfate, 1 M NaCl, 1% SDS and 5×10⁶ cpm ³²p labeled probe, at 65° C., with a final wash solution of 0.1×SSC and 0.1% SDS and final wash at 60° C.

According to another aspect of the present invention there is provided a method of increasing a level of a monosaccharide in a plant tissue, the method comprising the step of expressing a polypeptide having invertase activity, wherein the polypeptide is encoded by a polynucleotide at least 80% identical with SEQ ID NO:6 as determined using the BestFit software of the Wisconsin sequence analysis package, utilizing the Smith and Waterman algorithm, where gap weight equals 50, length weight equals 3, average match equals 10 and average mismatch equals −9.

The present invention successfully addresses the shortcomings of the presently known configurations by providing means with which the monosaccharide content of plant tissue or organ can be increased.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-i depict the chromosomal locations, sizes and identities of the 50 L. pennelli introgression lines (ILs) on chromosomes 1-12. The genetic map was constructed on the basis of 119 BC1 plants as described by Eshed and Zamir (1995) Mapped markers which are associated with the chromosome of a plant line, and markers not assayed on the BC1 map are placed according to their approximate positions based on Tanksley et al. Each line was probed with all the markers, and lines showing wild-species alleles are marked with bars to the left of the chromosome. c-L. esculentum, p-L. pennelli (Prior art).

FIG. 2 depicts the digenic interactions between unlinked QTLs. The values on the left and at the top of the Figures are the difference (in %) of each IL hybrid (ILH) from M82 according to Table 2 below. Values in bold are significant at p<0.05 (Dunnet's t test). Each histogram represents the difference (in %) of the hybrid heterozygous for the two introgression from the sum of the effect of the two individual ILHs for all traits measured (PW- plant weight, FM- fruit mass, B- brix, Y- total fruit yield, BY- the product of B and Y). Bars in white show no significant interaction and bars in light gray, gray and black indicate significant interactions of p<0.05, p<0.01 and p<0.001, respectively (prior art).

FIG. 3 depicts the distribution of the observed and expected numbers of pairs of introgressions showing simultaneous significant epistasis (p<0.05) for the traits: plant weight (PW), fruit mass (FM), brix (B) and yield (Y). The expected values were calculated on the basis of complete independence between traits and a mean epistatic rate of 0.28 for each trait (prior art).

FIG. 4 depicts the relationship between the expected an observed values for plant weight, fruit mass, brix, yield, and brix x yield of 45 hybrids of two ILs. Expected values were calculated on the basis or complete additivity of the effects of the individual ILHs. (prior art).

FIG. 5 depicts the fine mapping of linked QTLs for B and FM on the long arm of chromosome 2. The dark bars represent the L. pennelli chromosome segments introgressed into M82. Each point is the mean of the estimated introgression effect; bars represent the standard errors of the means. The mean phenotypic value of each line was determined as described in example 2 of the Examples section that follows (prior art).

FIG. 6 is a photograph depicting the fruit size of lines used for the mapping analysis of the linked QTL on chromosome 2 Top, IL2-5. Second row: left, IL2-5-1; right, IL2-6-1. Third row: left, IL2-5-3; center, IL2-6-6, right, IL2-6-4. Bottom, M82 (prior art).

FIG. 7 depicts an interaction between 1L9-2-5 and the year grown as expressed by plant weight (PW), fruit mass (FM) and brix (B). The values of IL9-2-5 and the hybrid ILH9-2-5 over the years 1995, 1996 and 1997 are expressed as the percent difference from the isogenic control M82 (Δ% of M82). Results for IL9-2-5 are indicated by the gray bars while results for ILH9-2-5 are indicated by the ladder bars. * above the bars denotes a significance difference (p<0.01) from the control and * in the d values represents a significant (p<0.05) dominance deviation of the heterozygous. For the traits showing no yearly dependence (alpha level=0.01) data from the three years was pooled to estimate a and d. The mean and the standard deviation values for M82 are indicated at the bottom of the Figure; PW- Kg, FM- g; B−%.

FIG. 8a depicts a genetic map of the IL9-2-5 introgression. The genetic distance in centimorgans (cM) is indicated between each pair of markers and is based on the F2 population. The genotype of IL9-2-5, IL9-2-6 and IL9-2-7 is represented by a hatched bar (L. pennelli) and an empty bar (L. esculentum). The border between the two bars is determined arbitrary between the two flanking markers.

FIG. 8b depicts the phenotypic effects of the IL9-2-5 (ladder bar), IL9-2-6 (black) and IL9-2-7 (white) hybrids compared to the control M82. * and ⁺ above the bars denote a significance difference (p)<(0.05, p<0.1, respectively) from the control. Each value represents the mean of eight plots.

FIG. 9 is a scatter plot depicting brix values of two isogenic hybrids, M82×line 202 (17 plants) and IL9-2-5×line 202 (8 plants). The center lines of the means (diamonds) are the group means. The top and bottom of the diamonds form the 95% confidence intervals for the means sp9—a novel tomato marker (SEQ ID NO:9 as a probe and EcoRV as a restriction enzyme).

FIG. 10 is a schematic depiction of the IL9-2-5 and IL-9-2-4 introgressions with respect to tomato chromosome 9.

FIG. 11 is a collection of scatter plots depicting the effects of brix 9-2-5 in the 3-year trial of the indeterminate (glasshouse) NILs. c- L. esculentum, p—L. pennelli. The homozygous IL (pp), containing segment of chromosome 9, is improved B by 27 percent over the control (ee) with partial dominance for the wild species segment (ep) (a=0.5, d=0.25, d/a=0.5). Black arrows and horizontal gray lines mark the mean values and the 99.9% confidence interval for each genotype.

FIG. 12 is a collection of scatter plots depicting the effects of Brix9-2-5 in the 3-year trial of the determinate NILs. ee, pp and ep represent NILs homozygous for the L. esculentum allele Brix9-2-5. NILs homozygous for the L. pennelli allele, and heterozygous NILs, respectively. Black arrows and horizontal gray lines mark the mean values and the 99.9% confidence interval for each genotype.

FIG. 13 depicts the fine-mapping and physical positioning of Brix.9-2-5 on chromosome 9. The upper portion shows the genetic linkage map (in cM) of the chromosomal region of Brix9-2-5, wherein the two end clones of BAC9IA4, 91N and 91S, are indicated in boxes. The mid portion shows the genetically ordered markers on BAC91A4 and the number of recombinants between them. The lower portion shows the recombination groups in the BAC. Each group in the lower portion is composed of families with a common introgressed segment and is represented by at divided bar of hatched (L. pennelli) and empty (L. esculentum) genomic segments. The borders between bars are arbitrarily drawn midway between markers positive and negative for the introgressed L. pennelli segment.

FIG. 14a depicts the nucleotide polymorphism (NP) and phenotypic analysis of 13 recombinant families of Brix9-2-5. Each NP is represented by a nucleotide number and the corresponding L. pennelli (top) and L. esculentum nucleotides. Full black bars denote a significant phenotypic effect (p<0.001). * denotes a verification of recombinant family 6 (−3%, nor significant) in an F4 generation.

FIG. 14b depicts the genomic structure of the Lin5 gene. Boxes depict exons and the arrows represent the recombination points for each of the individual recombinant families (numbered as in FIG. 14a), the nucleotide sequence of the L. pennelli Lin5 region spanning the QTL is presented by nucleotides 2301-2850, which are numbered to correspond to the start codon of Lin5. NILs between the two species are shown in bold and the codons for the 3 amino acids substitutions are underlined: positions 2403 (Asp in L. pennelli to Glu in L. esculentum), 2457 (Asp to Asn) and 2478 (Val to Leu); the intron sequence is depicted by outlined letters. Deleted nucleotides in the L. esculentum sequence are boxed and a four bp insertion (ATCT) following base 2735 is indicated by a a ^{v}. The 18-bp direct repeat is double underlined and the 7-bp repeats are marked with a wavy line. The start and stop codons of a hypothetical intraintronal open reading frame are denoted by dashed boxes.

FIG. 15 depicts the Lin5 exon-intron structure.

### DESCRIPTION OF THE PREFERRED EMBODIMENTS

The present invention is of isolated polynucleotides encoding novel plant invertases which can be used to increase the monosaccharide content in plants transformed therewith. Specifically, the present invention can be used to increase the monosaccharide content in plant tissues, such as, for example, fruits, leaves or roots by expressing at least one of the isolated polynucleotides which encode said novel plant invertases within the plant. The present invention is further of a novel plant expression regulatory element which can be used to increase the monosaccharide content in fruits of plants into which this regulatory element is genomically integrated in a site specific manner, especially in solanaceae.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Edible plant tissues, such as fruit, which store a high level of monosaccharides are a particularly sought after for both commercial processing and personal consumption.

As such, plant breeding techniques are often used by plant breeders in order to transfer such a desirable trait into cultivated species.

However, plant breeding techniques can only be used for related plant species and as such this desired trait cannot be transferred between unrelated plant species.

As such, the isolation of a gene or genes which are responsible for this trait is necessary such that recombinant techniques can be used to introduce this trait into a wide range of plants.

One family of genes which are responsible for monosaccharide generation in plants are the extracellular invertases.

Extracellular invertases enzymes are hydrolases, cleaving sucrose to glucose and fructose, which are transported into the cells. This activity maintains a gradient of assimilates, from the source parts of the plant, to the developing sink tissues. Cell wall invertases are synthesized as preproteins, with a long leader sequence which is cleaved off during transport and protein maturation. All known cDNA-derived amino acid sequences of invertases possess a signal peptide, required for entry into the endoplasmatic reticulum (ER) and, thus, into the secretory pathway. The mature peptide includes the NDPNG (SEQ ID NO:7) and WECPDF (SEQ ID NO:8) sequences which form the β-fructosidase motif and the catalytic site, respectively.

In tomato, the apoplastic invertase isoenzymes are encoded by a gene family comprising four members: Lin 5, Lin 6, Lin 7 and Lin 8 (Godt and Roitch, Plant Physiol. 115, 273-282, 1997). The published sequences of this gene family are mostly of the third and biggest exon of the gene, exon 3, and the full sequence of each of these genes remains undetermined.

As further detailed hereinbelow in Examples 3-5 of the Examples section below, while reducing the present invention to practice, a carefully planned approach using marker selected breeding of tomato plant introgression lines (ILs) enabled the determination of a narrow chromosomal region which is associated with the high level of monosaccharide accumulation (brix value) in L. pennelli fruits. Sequencing of a bacterial artificial chromosome (BAC) which includes this region has revealed the existence of two novel invertase genes which display identity to previously published Lin5 and Lin 7 partial cDNA sequences and which are termed herein as eLin5 and eLin7, respectively.

Sequencing of a cosmid clone from a L. pennelli genomic library revealed the full genomic sequence of the wild species allele which is termed herein as pLin5.

Comparison to L. esculentum sequences of the same chromosomal region has also revealed the existence of a sequence unique to the L. pennelli chromosomal sequence. This sequence which is 484 base pairs long spans a portion of the genomic polynucleotide sequence of pLin5 which includes a 3′ portion of exons 3, intron 3 and a 5′ portion of exon 4 of pLin5.

As further detailed in Example 5 of the Examples section, this sequence functions in regulating the expression of Lin5. As is further detailed in the Examples provided hereinbelow, the expression products of Lin5 transcript(s) function in contributing to the high brix value associated with L. pennellii nearly isogenic line(NIL) fruits.

Thus, according to one aspect of the present invention there is provided an isolated nucleic acid comprising a genomic, complementary or composite polynucleotide sequence encoding a polypeptide having an invertase activity in an apoplastic environment and an N terminal amino acid sequence serving for secretion into an apoplast.

As used herein in the specification and in the claims section that follows, the phrase “complementary polynucleotide sequence” includes sequences which originally result from reverse transcription of messenger RNA using a reverse transcriptase or any other RNA dependent DNA polymerase. Such sequences can be subsequently amplified in vivo or in vitro using a DNA dependent DNA polymerase.

As used herein in the specification and in the claims section that follows, the phrase “genomic polynucleotide sequence” includes sequences which originally derive from a chromosome and reflect a contiguous portion of a chromosome.

As used herein in the specification and in the claims section that follows, the phrase “composite polynucleotide sequence” includes sequences which are at least partially complementary and at least partially genomic.

The phrase “having an invertase activity in an apoplastic environment” so is used herein to distinct cellular invertases from those secreted into the apoplast. Plant invertases are characterized by their subcellular localization, their pH optima and their characterizing isoelectric point, pI. Intracellular invertase are characterized by acidic pH optima and low pI and are thought to be in the vacuole, whereas extracellular invertases are also characterized by acidic optima but a high pI that enables its bounding to the negatively charged cell-wall. Comparison of the known plant invertase genes revealed at least two distinguishing motifs: (i) Cell-wall invertases carry the amino acid Proline in their β-fructosidase motif (WECPDF, SEQ ID NO:8), as is compared to Valine in the vacuolar peptide; and (ii) in contrast to the cell wall invertases, the intracellular invertases contain an additional C-terminal extension, which might be involved in the vacuolar targeting of the protein.

According to one preferred embodiment of the present invention, the isolated nucleic acid encoding a polypeptide having an invertase activity is at least 80%, at least 85%, at least 90%, at least 95%, at least 98-100% homologous to SEQ ID NO:5 as determined using the BestFit software of the Wisconsin sequence analysis package, utilizing the Smith and Waterman algorithm, where gap creation penalty equals 8 and gap extension penalty equals 2.

As used herein the terms “homology” or “homologous” refer to the resemblance between compared polypeptide sequences as determined from the identity (match) and similarity (amino acids of the same group) between amino acids which comprise these polypeptide sequences.

In addition, or alternatively this isolated nucleic acid is at least 80%, at least 85%, at least 90%, at least 95% identical with SEQ ID NO:6 as determined using the BestFit software of the Wisconsin sequence analysis package, utilizing the Smith and Waterman algorithm, where gap weight equals 50, length weight equals 3, average match equals 10 and average mismatch equals −9.

According to another preferred embodiment or the present invention, the isolated nucleic acid is hybridizable with SEQ ID NOs:1, 4 or 6 under moderate to stringent hybridization conditions suitable for polynucleotides longer than 200 base pairs.

Hybridization under moderate hybridization conditions is effected by a hybridization solution containing 10% dextrane sulfate, 1 M NaCl, 1% SDS and 5×10⁶ cpm ³²p labeled probe, at 65° C., with a final wash solution of 1×SSC and 0.1% SDS and final wash at 50 or 55° C. whereas, hybridization under stringent hybridization conditions is effected by a hybridization solution containing 10% dextrane sulfate, 1 M NaCl, 1% SDS and 5×10⁶ cpm ³²p labeled probe, at 65° C. with a final wash solution of 0.1×SSC and 0.1% SDS and final wash at 60 or 65° C.

According to another preferred embodiment of the present invention, the isolated nucleic acid encodes a polypeptide which is as set forth in SEQ ID NO:5 or portions thereof having the invertase activity.

This polypeptide sequence which is designated herein as eLin5 (SEQ ID NO:5) includes a secretion signal sequence and is expected to have high invertase activity under apoplastic environment conditions.

According to another preferred embodiment of the present invention the isolated nucleic acid is as set forth in SEQ ID NO:6 or portions thereof.

According to another aspect of the present invention there is provided an isolated nucleic acid comprising a genomic, complementary or composite polynucleotide sequence encoding a polypeptide having an invertase activity, the polypeptide is at least 80%, at least 85% at least 90% at least 95% at least 98-100% homologous to SEQ ID NO:5, as determined using the BestFit software of the Wisconsin sequence analysis package, utilizing the Smith and Waterman algorithm, where gap creation penalty equals 8 and rap extension penalty equals 2.

According to a preferred embodiment the isolated nucleic acid of this aspect of the present invention is hybridizable with SEQ ID NOs:1, 4 or 6 under moderate to stringent hybridization conditions.

According to another preferred embodiment, the isolated nucleic acid of this aspect of the present invention is at least 80%, at least 85%, at least 90%, at least 95%, at least 98-100% identical with SEQ ID NO:6 as determined using the BestFit software of the Wisconsin sequence analysis package, utilizing the Smith and Waterman algorithm, where gap weight equals 50, length weight equals 3, average match equals 10 and average mismatch equals −9.

According to another preferred embodiment of the present invention the isolated nucleic acid of this aspect of the present invention is as set forth in SEQ ID NO:6 or portions thereof.

According to another aspect of the present invention there is provided a nucleic acid construct including any of the isolated nucleic acid sequences mentioned hereinabove.

The nucleic acid construct according to the present invention can be utilized to express the isolated nucleic acid within a plant, plant derived tissues, or plant cells either possessing a cell wall or not (protoplasts)

Thus, according to a preferred embodiment of the present invention, the nucleic acid construct further includes a promoter for regulating expression of the isolated nucleic acid in a sense or antisense orientation.

Numerous plant functional expression promoters and enhancers which can he either tissue specific, developmentally specific, constitutive or induced and which can be utilized by the construct of the present invention, some examples are provided hereinunder.

As used herein in the specification and in the claims section that follows the phrase “plant promoter” includes a promoter which can direct gene expression in plant cells (including DNA containing organelles). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric i.e., formed of portions of at last two different promoters.

Thus, the plant promoter employed can be a constitutive promoter, a tissue specific promoter, an inducible promoter or a chimeric promoter.

Examples of constitutive plant promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

Examples of tissue specific promoters include, without being limited to, bean phaseolin storage protein promoter, DLEC promoter, PTSβ promoter, zein storage protein promoter, conglutin gamma promoter from soybean. AT2S1 gene promoter, ACT11 actin promoter from Arabidopsis, napA promoter from Brassica napus and potato patatin gene promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, YNPS. prxEa, IIa h-sp17.7G4 and RD21 active in high salinity and OsnOtiC stress, and the promoters hsr203J and str246C active in pathogenic stress.

The construct according to the present invention preferably further includes all appropriate selectable marker such as for example an antibiotic resistance gene. In a more preferred embodiment according to the present invention the construct further includes an origin of replication.

The construct according to the present invention can he a shuttle vector, which can propagate both in E. coli (wherein the constrict comprises an appropriate selectable marker and origin of replication) and be compatible for propagation in cells, or integration in the genome, of a plant. The construct according to this aspect of the present invention can be, for example, a plasmid, a bacmid, a phagemid, a cosmid, a phage, a virus or an artificial chromosome.

There as various methods of introducing nucleic acid constructs into both monocotyledonous and dicotyledenous plants (Potrykus. I., Annu. Rev. Plant. Physiol., Plant. Mol. Biol. (1991) 42. 205-225; Shimamoto et al., Nature (1989) 338: 274-276). Such methods rely on either stable integration of the nucleic acid construct or a portion thereof into the genome of the plant, or on transient expression of the nucleic acid construct in which case these sequences are not inherited by a progeny of the plant

There are two principle methods of effecting stable genomic integration of exogenous nucleic acid sequences such as those included within the nucleic acid construct of the present invention into plant genomes:

(i) Agrobacterium-mediated gene transfer: Klee et al. (1987) Annu. Rev. Plant Physiol. 38:467-486; Klee anti Rogers in Cell Culture and Somatic Cell Genetics of Plants, Vol. 6, Molecular Biology of Plant Nuclear Genes, eds. Scholl, J., and Vasil, L. K., Academic Publishers, San Diego, Calif. (1989) p. 2-25; Gatenby, in Plant Biotechnology, eds. Kung, S. and Amtzen, C. J., Butteiorti Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, I. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment Klein et al. Bio/Technology (1988) 6:559-563: McCabe et al Bio/Technology (1998) 6:923-926; Sanlord, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plaint genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Following transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Transient expression methods which can be utilized for transiently expressing the isolated nucleic acid included within the nucleic acid construct of the present invention include, but are not limited to, microinjection and bombardment as described above but under conditions which favor transient expression and viral mediated expression wherein a packaged or unpackaged recombinant virus vector including the nucleic acid construct is utilized to infect plant tissues or cells such that a propargating recombinant virus established therein expresses the non-viral nucleic acid sequence.

Viruses that have been shown to be useful for the transformation of plant hosts include CaV, TMV and BV. Transformation of plants using plant viruses is described in (U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292, Takamatsu et al. EMBO J. (1987) 6:307-311; French el al. Science (1986) 231:1294-1297; and Takamatsu et al. FEBS Letters (1990) 269:73-76.

When the virus is a DNA virus, the constructions can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coal protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression nl the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant vital nucleic acid is capable of replication in the host, systemic spread in the host, and transcription or expression of foreign gene(s) in the host to produce the desired protein.

Thus, according to a preferred embodiment of the present invention the polynucleotide or nucleic acid molecule of the present invention further includes one or more sequence elements, such as, but not limited to, a nucleic acid sequence encoding a transit peptide, an origin of replication for propagation in bacterial cells, at least one sequence element for integration into a plant's genome, a polyadenylation recognition sequence, a transcription termination signal, a sequence encoding a translation start site, a sequence encoding a translation stop site, plant RNA virus derived sequences, plant DNA virus derived sequences, tumor inducing (Ti) plasmid derived sequences and a transposable element derived sequence.

According to another aspect of the present invention there is provided a method of increasing a level of a monosaccharide in a plant tissue, the method comprising the step of expressing in the plant tissue a polypeptide having invertase activity, wherein the polypeptide is at least 80%, at least 85%, at least 90% at least 95%, at least 98-100% homologous to SEQ ID NO:5 as determined using the BestFit software of the Wisconsin sequence analysis package, utilizing the Smith and Waterman algorithm, where gap creation penalty equals 8 and gap extension penalty equals 2.

The polypeptide according to this aspect of the present invention is preferably encoded by a polynucleotide which is hybridizable with SEQ ID NOs:1, 4 or 6 or a portion thereof under mild or stringent hybridization conditions as described above.

To effect expression, this polynucleotide sequence is preferably included in nucleic acid construct which also includes a promoter and selection markers as described hereinabove.

It will be appreciated that any of the transformation methods described hereinabove can be used to transform a plant or plant tissues with the above described construct, such that expression of the isolated nucleic acid according to any aspect of the present invention is effected.

Hybridization under mild hybridization conditions is effected by a hybridization solution containing 10% dextrane sulfite, 1 M NaCl, 1% SDS and 5×10⁶ cpm ³²p labeled probe, at 65° C., with a Final wash solution of 2×SSC and 0.1% SDS and final wash at 48° C.

According to another aspect of the present invention there is provided a method for determining whether fruits to be produced from solanaccae seeds or solanaceae seedling will contain an amount of monosaccharides above a predetermined threshold.

Thus, the present invention describes novel genes encoding apoplastic invertase isoenzymes which function either in combination or individually in elevating the monosaccharide of plants expressing sane.

A promoter sequence controlling the expression of Lin5 is also within is the scope of the present invention. Such a promoter resides upstream to Lin5 and its sequence is included in SEQ ID NO:1 (nucleotides 1-4849).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Mutual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perhal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); “Cell Biology: A Laboratory Handbook”, Volumes I-III Cellis, J. E, ed (1994); “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification aid Characterization—A Laboratory Course Manual” CSHL Press (1996); “An introduction to genetic analysis”—third edition, Suzuki et al., 1986 and “Molecular Dissection of Complex Traits, Paterson A H is 1998; all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

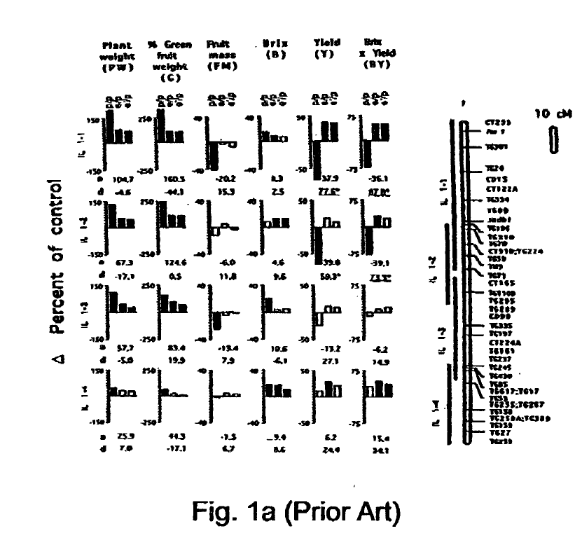

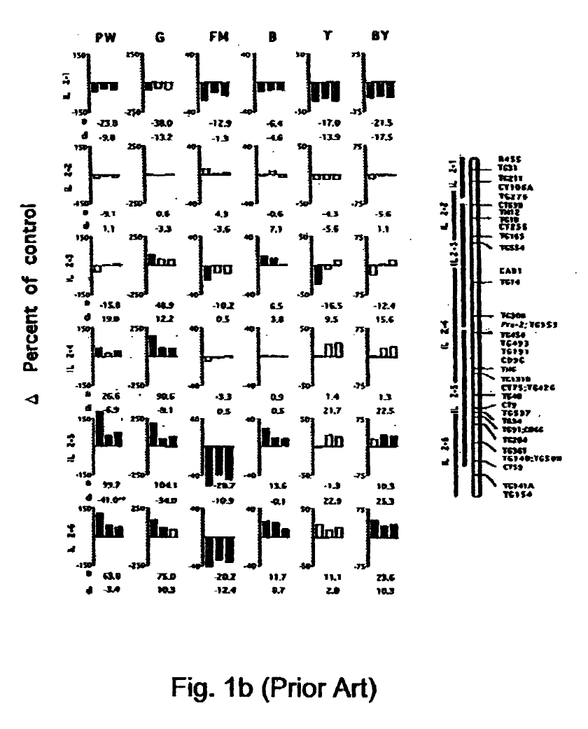

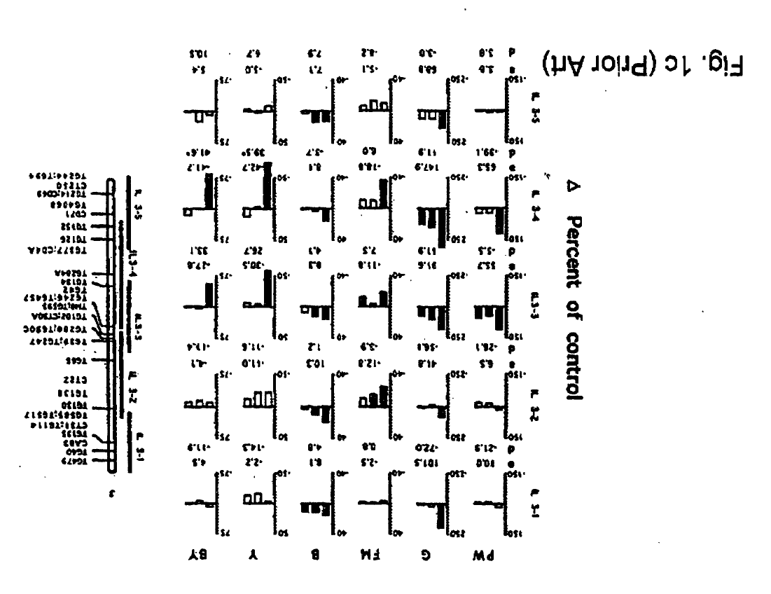

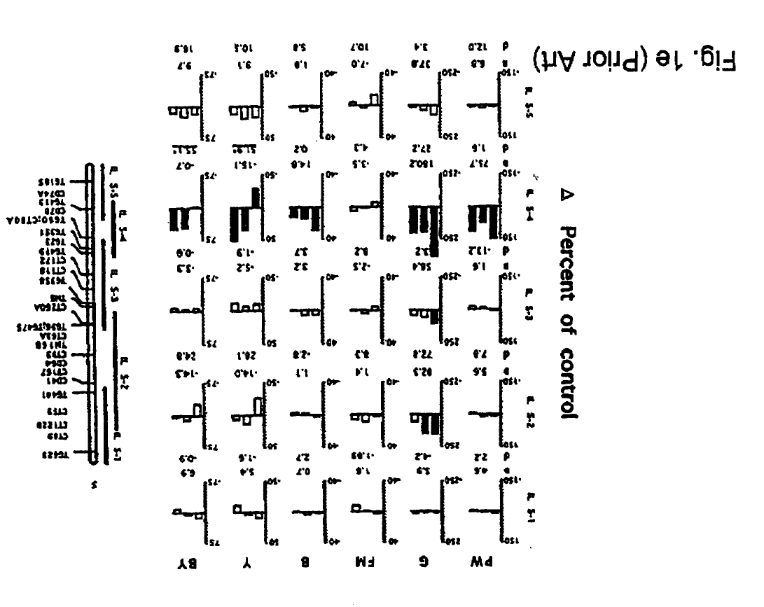

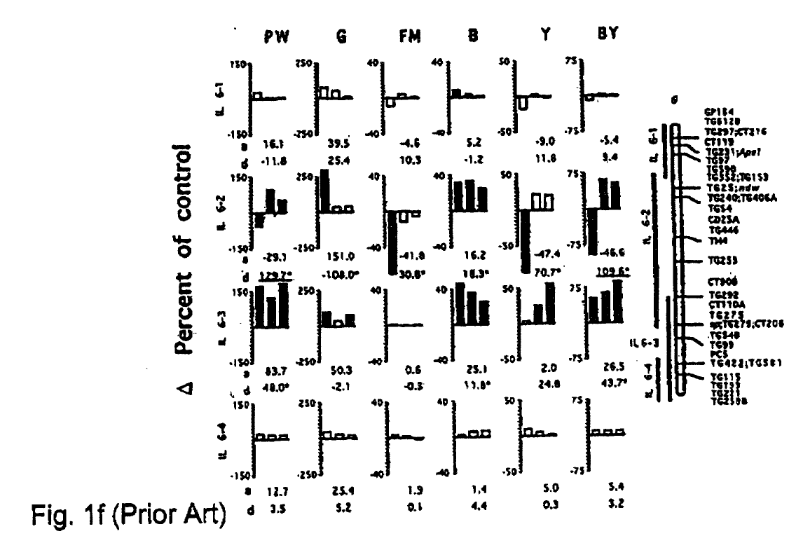

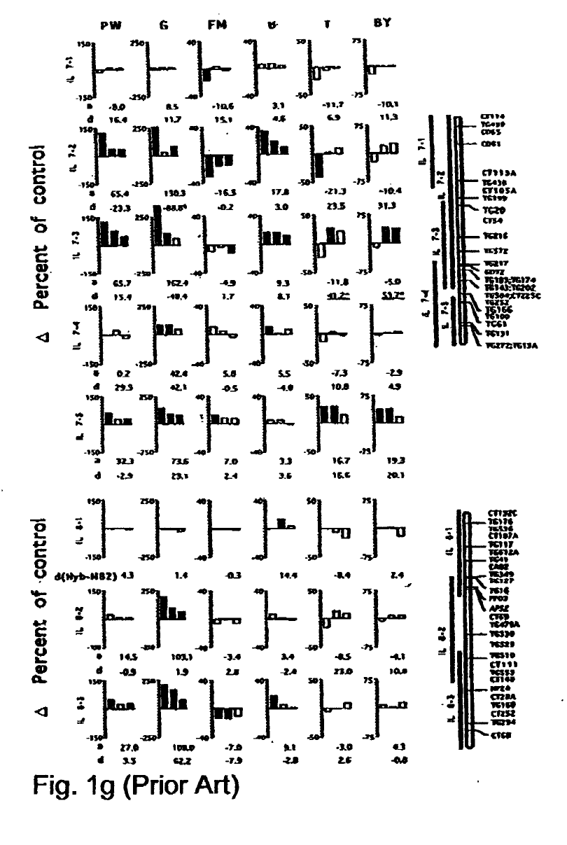

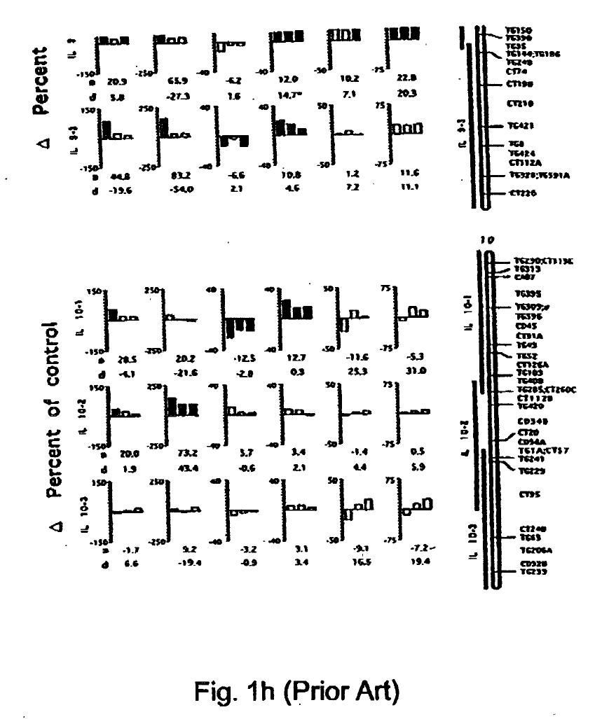

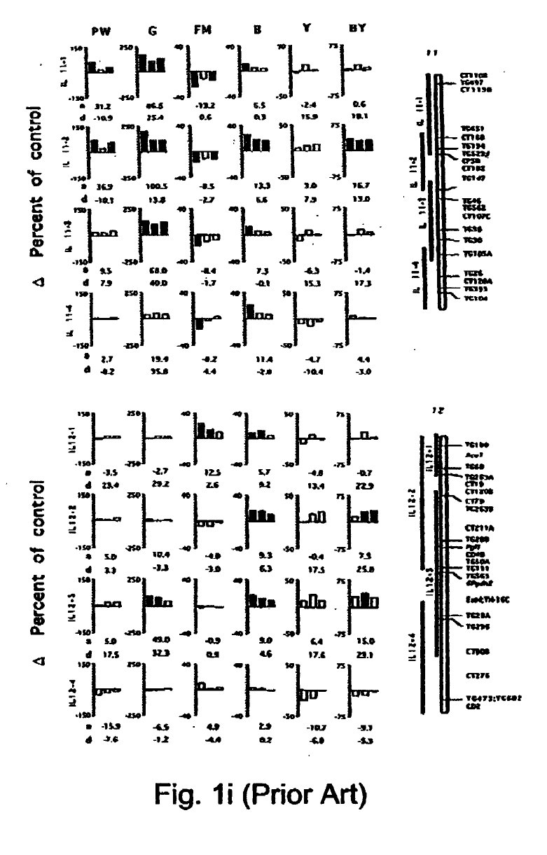

In previously published results (Eshed and Zamir, 1995, ibid) an L. pennelli introgression line (IL) population was designed in order to generate QTL-NILs. This IL population consisted of 50 lines, each containing a single homozygous restriction fragment length polymorphism (RFLP)-defined wild-species chromosome segment. Together these lines provided complete coverage of the tomato genome and a set of nearly isogenic lines (NILs) to their recurrent parent, the processing-tomato cultivar M82 (Rick et al., TGRC stock lists, Rep. Tom. Genet. Coop., 45, 53, 1995) (FIG. 1). The genetic assumption underlying the identification of QTL using the NILs was that any phenotypic difference between an IL and its nearly isogenic control plant is due to a QTL that requires on the chromosome segment introgressed from L. pennelli. The minimum number of p<0.05-significant QTL affecting a trait in the ILs was calculated on the basis Of the following assumptions: (i) each IL affecting a quantitative trait carries only a single QTL; and (ii) two overlapping introgressions with a significant effect on a trait (in the same direction relative to the control) carry the same QTL.

Therefore, in the ILs, the maximum number of detectable QTLs is approximately 30. Despite this limitation, twice as many QTLs responsible for fruit mass (FM) were identified as compared to the previous populations (see Table 1 below). The sensitivity of the ILs in identifying QTLs was even more pronounced for brix (B), where two to six times as many QTLs were identified as compared to the other populations.

TABLE 1
The number of significant effects (p < 0.05) of wild species QTLs on FM and B

| | population | population | No of | No of | |
|---|---|---|---|---|---|
| Species | structure | size | FM-QTLs | B-QTLs | Reference |
| L. chmielewskii | BC1 | 237 | 6 | 4 | Tanksley et al. |
| | | | | | Genetics, |
| | | | | | 232, 1141, 1992 |
| L. cheesmanii | F2 | 350 | 7 | 4 | Patterson et al |
| | | | | | Genetics, |
| | | | | | 127, 181, 1991 |
| L. | BC1 | 257 | 7 | 3 | Grandillo et al |
| pimpinellifolium | | | | | Theor. Appl. Genet., |
| | | | | | 90, 225, 1996 |
| L. cheesmanii | RI | 97 (6 reps.) | 12 | 14 | Goldman et al |
| | | | | | Theor. Appl Genet., |
| | | | | | 90, 925, 2995 |
| L. pennellii | IL | 50 (6 reps.) | 28 | 23 | Eshed and Zamir |
| | | | | | 1995, ibid |

Using the L. pennelli ILs, QTLs were mapped to various chromosome segments originating from the wild species However the effects associated with an introgressed segment could be due to the existence of one or more loci.

A 60-cM segment on the long arm of chromosome 2 was responsible for a 60% reduction in FM (in homozygotes) relative to the control, M82. This chromosomal region apparently harbors QTLs responsible for FM, which are common to a number of wild tomato species (Alpert et al. Theor. Appl. Genet., 91, 994, 1995).

Fine-mapping analysis of recombinant lines for that region identified three linked loci with a similar effect on FM; two of which were placed on a 3 cM interval. Finer mapping may reveal additional FM QTL in these regions.

Quantitative effects which appear to be associated with a single locus were inferred from cases of rare transgressive segregation. Using the ILs, 18 QTLs responsible for FM were identified but in only two cases (IL7-5 and IL12-1-1 with introgression sizes of 15 and 4 cM, respectively) alleles of the small-fruited wild species were associated with larger fruits. These effects were consistent in trials conducted in different years and genetic constitutions (Eshed and Zamir, 1996, ibid).

Several features of the IL population contributed to its efficiency in detecting QTLs, even in cases when only a few replicates of each genotype were evaluated.

(i) The lines contained single RFLP-defined introgressions, some of which produce effects of relatively large magnitude in which most of the phenotypic variation between the NILs is associated with the introgressed segment.

(ii) The permanent nature of the lines enabled testing of the introgression effects in different years. The results obtained showed high reproducibility of the effects of the QTL which were mapped to the different introgressed chromosome segments.

(iii) Elimination of the “overshadowing effect” of major QTLs enabled to detect minor QTLs (a major QTL contributes to large phenotypic variation, thereby masking the effects of other QTLs segregating in the same population)

(iv) Elimination of epistatic interactions between unlinked QTLs.

(v) The simple statistical procedure relied on comparison with a common control and is therefore less affected by experimental error.

### Gene Actions Revealed by QTL Studies

A gene action of the QTL detected was determined using the IL population described above by comparing the homozygous ILs to hybrids of the ILs with the recurrent parent.

The FM and brix qualities were determined by QTLs which were intermediates between additivity and dominance. This mode of inheritance is in agreement with results obtained by analysis of an F2 generation (Paterson Genetics, 127, 191, 1991). In contrast, fruit yield (Y) was strongly associated with overdominance, whereby some of the heterozygous ILs bad higher values relative to their corresponding homozygous parents.

Detailed mapping analysis of a chromosome 1 introgression which showed overdominance for Y suggested the existence of two cis loci with opposing effects. This result was therefore consistent with the pseudo-overdominance model for heterosis (Crow Heterosis, Gowen, J. W., Eds., Iowa State College Press. Ames, Iowa, 1952). However for the other heterotic introgressions, including dw-1, the issue of the mode of gene action for heterosis is still unresolved. It is interesting to note that the wild species used for the tomato mapping studies were highly inferior to the cultivated variety with respect to Y, yet chromosome segments from these species contribute to the increased Y of commercially grown varieties. This transgressive segregation is frequent for Y and for seedling morphological traits, whereas for FM and brix transgression was rare (De-Vicente et al Genetics, 134, 585, 1993).

### Reproducibility of the Effects of an Identified QTL

Mendelian factors underlying quantitative traits in an interspecific tomato cross were compared in F2 and F3 generations of the same population (Paterson Genetics, 127, 181, 1991). Of 11 FM QTL identified in both generations in a trial conducted in California, six were significant both in F2 and F3. Of the five B QTLs, two were significant in both generations. Differences between generations can result from interactions with the environment and/or may indicate that the resolution power of such populations is limited to QTLs with large effects. In contrast, of 33 yield-associated QTLs identified in a two-year trial of selected ILs, 28 were significant in both experiments (Fished and Zamir 1996, ibid).

Association between QTL-NILs and the introgressed segment The use of the L. pennelli Il s to identify QTLs is based on RFLP results which indicated that each line contains a single wild-species introgression. However, some of the lines may include small unidentified introgressions, and these segments may be responsible for the observed phenotypic effects. To test whether the difference between the IL and its nearly isogenic control lies solely in the introgressed segment, a simple experiment was performed using eight selected ILs. An F2 resulting from a cross between each IL and M82 was subjected to RFLP analysis, and plants homozygous for the cultivated-tomato chromosome segment were compared quantitatively to M82. In no case were any differences detected, indicating that the observed phenotypic differences (which were verified using the plants carrying the L. pennelli introgressions) are due to the mapped chromosome segment.

### EXAMPLE 2

### Epistatic Interactions

The study described in Example 1 (Eshed and Zamir, 1995, ibid) served as a basis for testing epistatic interactions between QTLs. Thus, 10 ILs were so selected, some of which include QTLs that affect the measured traits in the heterozygous condition in various directions relative to the control the results obtained were reported.

The ten homozygous ILs were crossed in a half diallele mode and the phenotypic values of the 45 double heterozygotes were compared to the respective single heterozygous ILs and M82. The results which were previously reported by Eshed and Zamir (1996, ibid) indicate that QTL epistasis is prevalent and is generally less than additive.

Phenotype of the Selected Single Introgression ILHs:

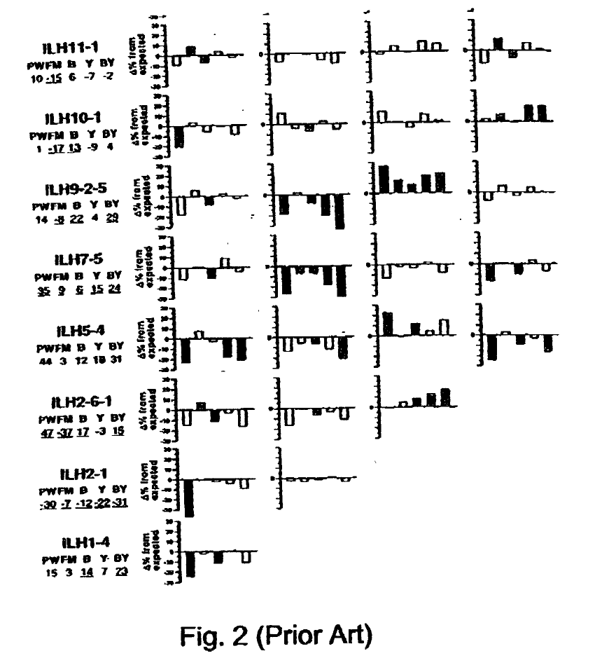

In the complete IL population composed of 50 introgression line hybrids (ILHs) which was analyzed for five yield-associated traits, 81 of the 250 ILH×trait combinations (32%) were significantly different from the isogenic controls (p<0.05). For the subset of the 10 ILHs selected for the interaction study, 30 of the 50 combinations (60%) differed significantly from the control (FIG. 2). This comparison indicates that the 10 ILHs were enriched for QTLs affecting the measured traits.

In this previously reported study, of the 10 ILHs (using the same experimental error), 28 of the 30 significant effects were consistent between the two experiments (Table 2 below; Y for ILH1-4 and BY for ILH2-6-1 were not significantly different from the control).

TABLE 2
Mean phenotypic values of M82 and the IL hybrids heterozygous for single introgressions

| | | Number | plant | fruit | | | brix × |
|---|---|---|---|---|---|---|---|
| | Introgressed | of | weight | mass | (kg) | Yield | yield |
| Genotype | region ‘a’ | replicates | (kg) | (g) | brix (0) | (kg) | (g) |
| M82 | none | 79 | 1.82 ± 0.44 | 56.1 ± 4.9 | 4.54 ± 0.40 | 9.18 ± 1.54 | 417 ± 82 |
| ILH1-1^{b} | 1(CT233-TG71: | 26 | 3.56 ± 0.84* | 48.6 ± 6.5* | 5.23 ± 0.48* | 11.15 ± 2.34* | 580 ± 114* |
| | 58 cM) | | | | | | |
| ILH1-4 | 1(TG245-TG259; | 23 | 2.10 ± 0.44 | 58.1 ± 5.4 | 5.16 ± 0.39* | 9.84 ± 1.60 | 507 ± 83 |
| | 35 cM) | | | | | | |
| ILH2-1 | 2(R45S-TG276: | 26 | 1.27 ± 0.32* | 52.4 ± 54* | 4.01 ± 0.33* | 7.19 ± 1.64* | 289 ± 72* |
| | 16 cM) | | | | | | |
| ILH2-6-1^{c} | 2(TG91-CT59: | 26 | 2.68 ± 0.46* | 35.2 ± 4.5* | 5.30 ± 0.44* | 8.95 ± 1.81 | 474 ± 99 |
| | 14 cM | | | | | | |
| ILH5-4 | 5(TG351- | 25 | 2.62 ± 0.59* | 57.9 ± 7.1 | 5.07 ± 0.31* | 10.85 ± 2.34* | 551 ± 127* |
| | TG413; 16 cM) | | | | | | |
| ILH7-5 | 7(TG61- | 26 | 2.46 ± 0.46* | 61.5 ± 6.3* | 4.83 ± 0.33* | 10.52 ± 1.45* | 509 ± 87* |
| | TG131A; 15 cM) | | | | | | |
| ILH9-2- | 9(CT283A- | 25 | 2.09 ± 0.47 | 51.7 ± 6.3* | 5.52 ± 0.26* | 9.58 ± 1.92 | 532 ± 122* |
| 5^{c} | TG10; 9 cM) | | | | | | |
| ILH10- | 10(TG230- | 24 | 1.84 ± 0.33 | 46.5 ± 5.5* | 5.11 ± 0.31* | 8.38+E ± 1.60 | 428 ± 81 |
| 1 | TG285; 37 cM) | | | | | | |
| ILH11- | 11(TG497- | 26 | 2.06 ± 0.47 | 47.5 ± 3.6* | 4.79 ± 0.41 | 8.50 ± 1.65 | 406 ± 73 |
| 1 | TG523; 27 cM) | | | | | | |
| ILH12- | 12(TG180 | 27 | 1.81 ± 0.32 | 63.3 ± 5.4* | 4.70 ± 0.37 | 8.96 ± 1.21 | 422 ± 69 |
| 1-1^{c} | ACO-1; 4 cM) | | | | | | |

Mean phenotypic values and standard deviations of M82 and the ILH that participated in the diallele crosses. All means were compared to M82 and the ones marked with * are significantly different (Dunnet's t-rest, p < 0.05). Underlined mean values indicate a significant interaction with year (1993 vs., 1995; 0.01 < p < 0.05).
^{‘a’}- The introgressed regions in the ILHs is indicated by chromosome number, the markers flanking the introgression and its size in cM according to Tanskley et al. (1992) Genetics 132:1141-1160.
^{b}- ILH - Hybrid of ILs crossed with M82.
^{c}- Interaction with year was based on unpublished results from a 1994 trial.

The effects of ILH7-5 on PW, FM and brix were found to be significant as compared to other previously reported studies. This significance was probably due to the larger number of replicants tested (25 as compared to 6 in previous studies). Significant ILH by year interactions (p<0.05) were detected for four of the 50 comparisons (Table 2). These four comparisons were not significantly different front the control in either of the years. These results indicate a high overall reproducibility of the experimental system in different years of growth.

Interactions Between Unlinked Introgressions:

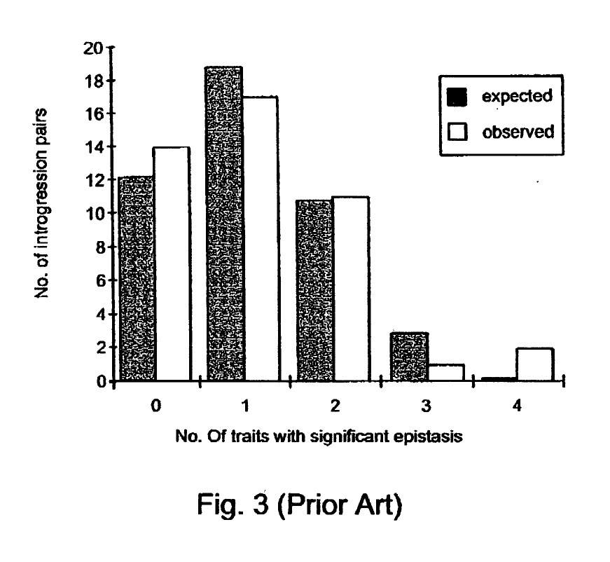

The null hypothesis for the interaction analysis was complete additivity of the effects of the single introgression ILHs. Any significant deviation from complete additivity was considered as epistasis (FIG. 3).

For example, ILH1-1 increased PW by 95% compared to M82; ILH12-1-1 reduced PW by 1% compared to M82. The expected phenotype for the hybrid between the two homozygous ILs (IL1-1 and IL12-1-1) is a 94% increase in PW relative to M82. The observed PW for the hybrid heterozygous for the two introgressions was 76% higher than M82, indicating a significant interaction (p<0.05).

Of the 225 possible interactions (45 hybrids×five traits) 59, 28 and 12 were significant at the p<0.05, p<0.01 and p<0.001level respectively (FIG. 3). These values are much higher than that expected by chance alone.

To further characterize the nature of the interaction, the double-heterozygous combinations were divided into four groups based on the performance of the single ILHs (Table 3 below).

TABLE 3
Frequency of significant interactions (p < 0.05) between
unlinked L. pennellii introgressions

| | | | | Total | | |
|---|---|---|---|---|---|---|
| Interacting | Plant | Fruit | | fruit | brix × | |
| QTL types^{a} | weight | Mass | brix | yield | yield | Sum |
| Sig—Sig (same | 3^{b}/6^{c} | 8/16 | 12/21 | 1/3 | 5/15 | 29/61 |
| direction) | | | | | | |
| Sig—Sig (opposite | 2/4 | 1/12 | 3/7 | 0/3 | 1/6 | 7/32 |
| direction) | | | | | | |
| Sig—NonSig | 5/25 | 2/16 | 4/16 | 5/24 | 2/21 | 18/102 |
| NonSig—NonSig | 1/10 | 0/1 | 0/1 | 3/15 | 1/3 | 5/30 |
| Sum | 11/45 | 11/45 | 19/45 | 9/45 | 9/45 | 59/225 |

^{a}QTLs were classified according to the significance and the direction of their effects relative to M82.
^{b}Number of significant interactions.
^{c}Number of tested combinations of two L. Pennellii introgressions.

As is shown by Table 3, of 61 tested introgressions between significant QTLs (same direction), 29 (48%) were significant (p<0.05) indicating that the interactions between two significant QTLs of L. pennelli affect a trait in the same direction.

Among 32 introgressions between significant (opposite) QTLs, seven (22%) significant interactions were detected indicating that the interaction between two significant QTLs of L. pennelli affect the trait in opposite directions. Six of these interactions involved crosses with 1L2-1 for PW, B and BY. The IL2-1 line carries the plciotropic QTL which affected all of these traits. The seventh interaction, in this group involved that of IL12-I-1 with IL-10-1 for FM, where IL12-1-1 showed marked transgressive segregation for this trait (Table 2).

Among 102 introgressions between significant and non-significant QTLs 18 (18%) interactions were significant.

Among the 30 introgressions between non-significant QTLs, five (17%) significant interactions were found. Overall, 26% (59/225) of the various L. pennelli introgressions showed significant interactions and the proportion of epistatic effects was highest for significant same direction QTLs.

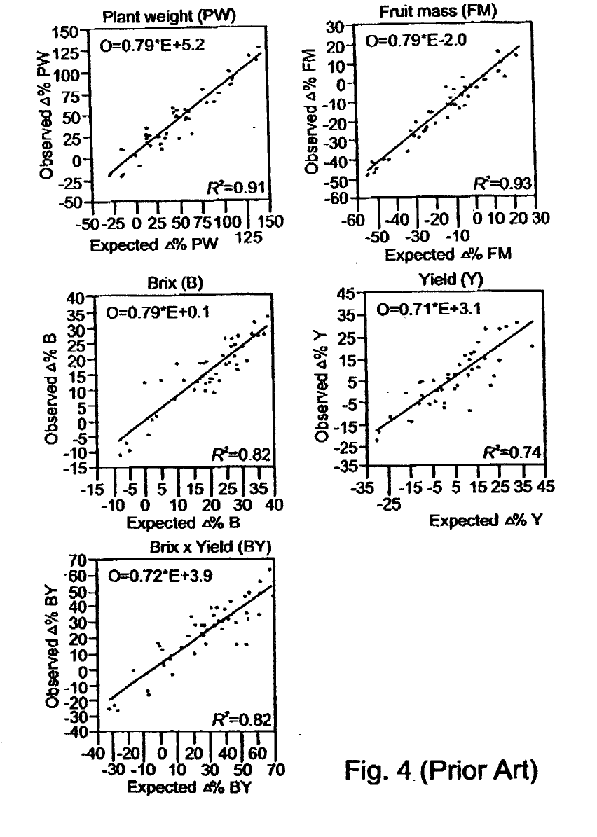

To search for general trends in the interaction of QTLs, the observed values of the 45 double-heterozygous hybrids were plotted against their expected values (FIG. 4).

For all five traits highly significant linear regressions were found, indicating the overall additivity of the effects of the independent introgressions. Assuming complete additivity between the effects of the combined individual introgressions one would expect a regression with a slope of 1. The slopes of the lines for the five traits were significantly lower than 1 (ranging from 0.71-0.79), indicating average combined effects which are less than additive

To further examine the less than additive trend revealed by the regression analysis, only the cases of epistasis between significant QTL affecting the traits in the same direction were examined irrespective of whether the QTL originated from L. pennelli or L. esculentum.

Twenty-nine epistatic interactions between L. pennelli introgressions were detected. In all cases, the observed means for the double heterozygous ILHs were significantly lower than the values expected on the basis of an assumption of complete additivity. Seven of the interactions of QTL affecting the trait in the same direction involved L. pennelli introgressions with L. esculentum alleles. In these cases (row 2 of Table 3), the L. pennelli introgressions affected the trait in an opposite manner to that expected according to the parental phenotype (transgressive QTL). Six of the seven interactions were less than additive; the only exception was PW for the hybrid of IL1-1×IL2-1 In this case the double heterozygous hybrid for the QTL acting in the same direction (ILH1-1) showed a higher mean value than the sum of the two independent QTL (M82 and IL2-1××IL1-1). Overall, 35 of the 36 interactions (97%) showed less than additive interactions.

Interactions Between Linked QTL (Chromosome 2):

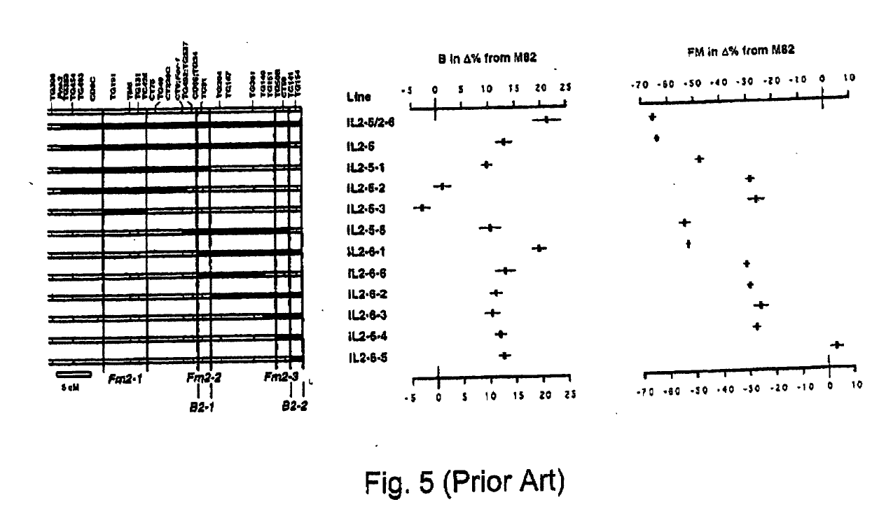

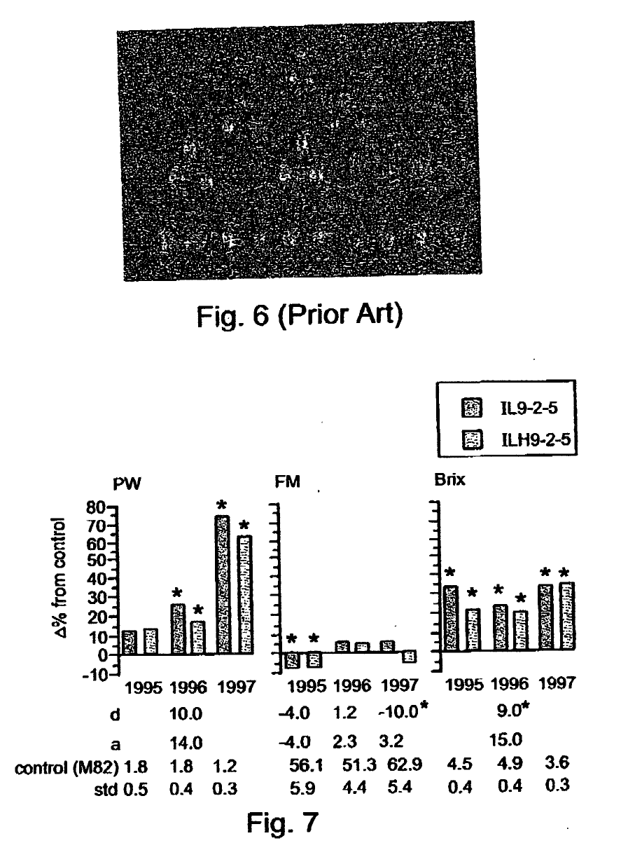

Twelve homozygous ILs with different introgression sizes in chromosome 2 were evaluated for FM and J. Since 10 of these lines were previously tested (Eshed and Zamir 1995, ibid) and no significant interactions between year and IL were detected, the results from the two years were pooled. Based on the overlapping recombined chromosome segments and the phenotypic value of each of the ILs, two B QTL and three FM QTL, responsible for a similar reduction in fruit mass, were mapped (FIGS. 5-6). After determining the positions of these QTL, the lines were classified according to their postulated genotypes (Table 4).

TABLE 4
Interactions of linked QTLs responsible for brix and fruit mass

| | Mean brix (B) | Mean brix | P value of |
|---|---|---|---|
| Genotypic group^{a} | in Brix units | Δ % from M82 | interaction |
| No QTL^{b} | 4.47 | −0.2 | |
| B2-1 | 5.00 | 11.7 | |
| B2-2 | 4.98 | 11.6 | |
| B2-1/2-2 | 5.37 | 19.9 | 0.03 |
| | Mean Fruit mass | Mean FM | P value of |
| Genotypic group^{a} | (FM) in grams | Δ % from M82 | interaction |
| No QTL^{b} | 59.5 | 0.6 | |
| Fm2-1 | 43.0 | −27.3 | |
| Fm2-2 | 41.0 | −30.7 | |
| Fm2-3 | 42.7 | −27.8 | |
| Fm2-1/2-2 | 30.7 | −48.2 | 0.009 |
| Fm2-2-/2-3 | 28.0 | −52.7 | 0.03 |
| Fm2-1/2-2/2-3 | 21.0 | −64.6 | <0.0001/<0.0001^{c} |

^{a}genotypic groups were pooled on the basis of the fine mapping analysis presented in FIG. 5.
^{b}M82 was included in this group, which includes lines without an L. pennellii QTL which affects this trait.
^{c}The two tested interactions were Fm2-1 × Fm2-2/2-3 and Fm2-3 × Fm2-1/2-2

Epistasis for B and FM was tested by comparing the means of the pooled genotypic groups. The single interaction for B was significant and the sum of the effects of the single QTL was higher than the mean value of the lines carrying both QTL. The four different tests for FM QTL interactions were significant: two of them examined the combined action of a single QTL and two examined a single QTL and the remaining pair. The average diminishing effect for two QTL was 8.5% compared to 16.2% for interactions involving the three QTL (Table 3). This result suggests that the effect of the less than additive epistasis is increased (i.e. the effects are further diminished) when more QTL are involved.

The nearly isogenic nature of the IL population utilized by this study allows the identification of twice as many QTL affecting FM and B as in other interspecific studies in tomato (Eslied and Zamir, 1995, ibid). The isogenic nature of the IL population is also responsible for the ability to determine epistasis of QTL through the design of experiments with balanced representation of the different genotypes. Nearly isogenic lines were previously demonstrated to be very efficient for the detection of epistasis of QTL in Drosophila (Long et al. 1995, Genetics 139:1273-1291) and maize (Doebley et al. 1995, Genetics 141:333-346). In conventional segregating populations (F2/F3, DC and recombinant inbreds) all the QTLs which affect the trait are segregating QTLs. Assuming that the less than additive mode of epistasis detected in this study is common to other tomato crosses, this interaction would reduce the effect of individual QTLs. As a consequence, the number of significant QTLs would be underestimated. Less than additive interactions among QTL ensure that the “loss” of an allele affecting a fitness trait will have a minimal effect on the phenotype and that canalization will be achieved.

Contrary to past QTL mapping studies that uncovered little evidence for epistasis, QTL epistasis is an important component in determining the phenotypic value for traits showing continuous variation (Table 3). Of the 93 combinations of pairs of significant QTLs, 39% were epistatic at a significance level of p<0.05. Moreover, a higher frequency of epistasis than expected by chance alone was detected for L. pennelli chromosome segments that individually did not affect the traits (17%).

Thus, the prevalence of epistasis uncovered by this study is consistent with the numerous classical studies of quantitative traits and breeding that show significant overall epistatic effects for quantitative traits detected through biometrical genetics.

### EXAMPLE 3

### Separating the Positive Trait for High Brix Value from the Negative Traits of Percentage Green Fruit Yield and Internodes Length Through Marker Assisted Selection

As is described in Examples 1 and 2, the hybrid plants obtained from introgressing L. pennelli into a L. esculentum genetic background detected numerous QTLs associated with traits such as brix (B) and fruit mass (FM). However, these studies failed to isolate the QTL associated with brix from other QTLs which arc associated with negative traits such as high percentage of green fruit yield and long internodes.

As such, while reducing the present invention to practice a hybrid plant (IL9-2-5) resultant from these studies was further introgressed into the genetic background of an L. esculentum cultivated tomato variety (M82) in efforts to isolate the QTL associated with brix from other QTLs responsible for these negative traits which are present in IL9-2-5, to thereby obtain a plant line bearing fruits characterized by a high sugar content (high brix value) while being otherwise similar in phenotype to a cultivated tomato.

To estimate the phenotypic variation associated with high brix value, near isogenic plants derived from self crossing of M82 and IL9-2-5 and hybrids generated from crossing M82 and IL19-2-5, were evaluated over a three year period. FIG. 7 presents the means of the tested genotypes for total soluble-solids (brix, B), plant weight (PW) and fruit mass (FM).

The 9-2-5 (chromosome 9) introgression was responsible for a significant reduction (10%) in FM in 1995 while in the following years its effects were not significant. The effect of the introgression on B was consistent between the different years; the introgression significantly increased B from 20 to 32 percent relative to the control showing partial dominance (d/a=0.64). In 1995, the introgression increased PW by ten percent compared to 70 percent in 1997; yet, the effect of the introgression on B was similar in these two years, indicating that PW is not involved in the major pathway affecting B. Hybrid high brix value plants that carried the introgression were more vegetative, with longer internodes and the ripening of the fruit lasted a longer period (late variety) and as such are of little commercial value.

In order to generate hybrids characterized by a uniform and early ripening, a good cover of the fruit and a high brix value, which hybrids are of high commercial value, it was decided that further narrowing of the 9-2-5 introgression chromosomal region (9 cM) must be effected in order to isolate the brix QTL.

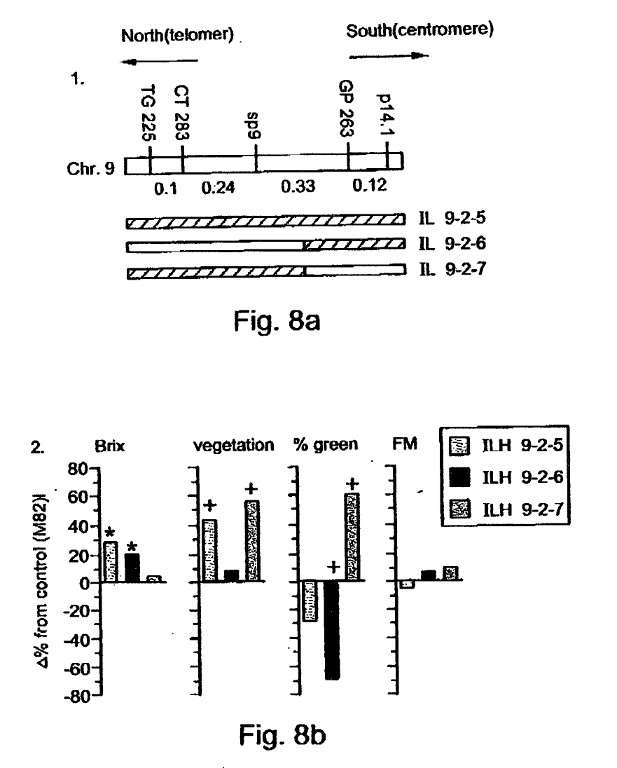

In order to verify if the increase in brix and the negative effects described above which are associated with the 9-2-5 introgression are due to linkage drag or are simply pleiotropic effects of the QTL, sub-lines of IL9-2-5 (IL9-2-6 and IL9-2-7) were generated by selfing the IL9-2-5 hybrid and screening for recombinants in the introgression. IL9-2-6 and IL9-2-7 carried the south (in direction of the centromere) and north (in direction of a telomere) part of the introgression, respectively (FIG. 8a). Plants of M82 and hybrids generated from crossing M82 with the IL9-2-5, IL9-2-6 and IL9-2-7 plant lines (termed ILH9-2-5, ILH9-2-6 and ILH-9-2-7, respectively) were planted in a commercial stand and evaluated for B, FM, vegetation and % of green fruit yield as a parameter for the uniformity of the ripening. FIG. 8b presents the mean effects of the tested hybrids as is compared to the control tomato plant M82. l he short introgressions of IL9-2-7 showed the “negative” phenotype of IL9-2-5 with high vegetation, longer internodes and late maturity, but had no significant effect on B. IL9-2-6 had a significant increasing effect on B with a reduced vegetation and an early and uniform ripening. The three hybrids had no significant effects on the FM.

Thus, these results place the brix QTL in the south part of the 9-2-5 introgression (FIG. 8a). A hybrid plant (ILH9-2-6) generated from introgressing IL9-2-6 in the M82 genetic background is characterized by fruit having an increased sugar content (B) similar to that of the IL9-2-5 hybrid plant line, without the undesired traits found in IL9-2-5 which are generated by genes situated in the northern part of the 9-2-5 introgression (9-2-7).

### EXAMPLE 4

The study described in Example 3 which was conducted as part of the present invention and previously published studies described in Examples 1 and 2, were performed in a genetic background of determinate tomato lines that were specifically developed for the processing tomato industry (M82). These plants are suitable for “once over” machine harvest due to homozygosity for the recessive mutation sp (self pruning) which modifies the developmental program of the shoot such that growth is terminated after the production of two consecutive inflorescences.

The wild species (green-fruited) and greenhouse cultivated tomatoes are indeterminate (Sp¹ where the shoot follows a uniform developmental program of three leaves and an inflorescence throughout the growth (Pnueli et al. 1998, Development 125:1979-1989). Indeterminate greenhouse tomatoes require different agricultural practices as is compared to determinate varieties and therefore constitute a fundamentally different genetic background to test the effect of the brix QTL.

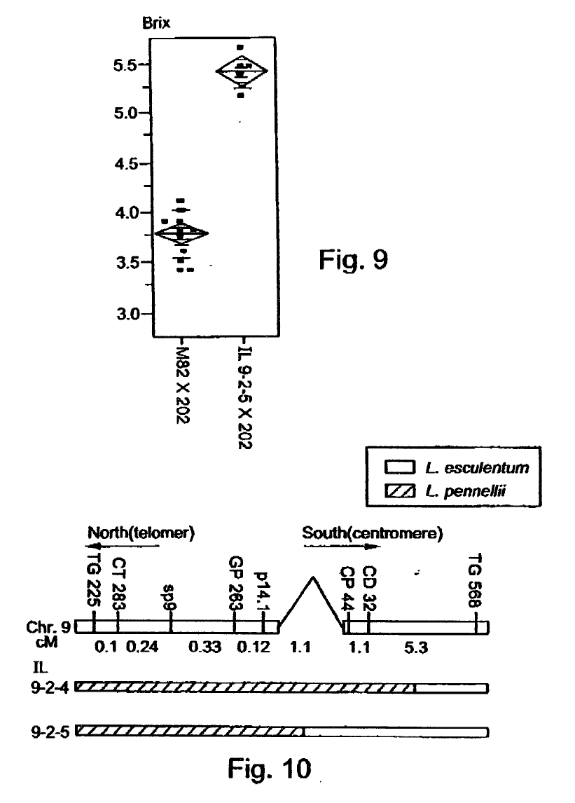

M82 and IL9-2-5 were crossed with an indeterminate greenhouse line (202) and the two nearly isogenic indeterminate hybrids were grown in the greenhouse and evaluated for B. The introgression was responsible for a 40 percent increase in B with a separation of the values into discrete groups (FIG. 9). This result gave a motivation to develop NILs for the chromosome 9 introgression in the genetic background of line 202.

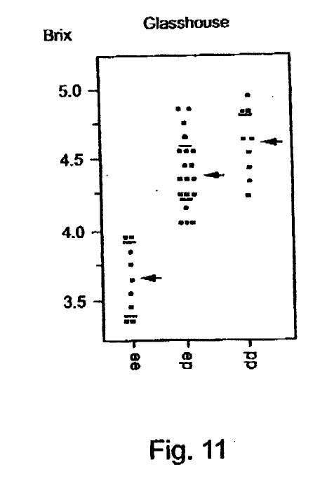

The initial material for the introduction of the brix QTIL into indeterminate background was the IL9-2-4 introgression line (FIG. 10). This introgression extends to the south of the chromosome beyond the 9-2-5 introgression. This line was selected since it was observed that recombinants are more efficiently obtained when long introgressions are used in the marker assisted selection. After five marker-assisted backcrosses the selfed generation of a BC5 plant that was heterozygous for the introgression was grown and the segregating population was subjected to RFLP analysis. The results were highly consistent between the determinate and indeterminate backgrounds (FIG. 11), the homozygous NIL, containing segment of chromosome 9, improved B by 27% over the control with partial dominance for the wild species segment (a=0.5, d=0.25, d/a=0.5). Very similar results were obtained in another growing season (data not presented) confirming that the observed effects were independent of environment in the greenhouse.

Thus, a major brix associated QTL was introgressed into a genetic background of an indeterminate greenhouse tomato (202) thus yielding plants which are high in brix and which in all other aspects are similar in phenotype to this indeterminant greenhouse tomato line. In addition, the resultant tomato line does not display the undesirable self pruning trait inherent to determinate tomato lines specifically developed for the processing tomato industry (M82).

### EXAMPLE 5

Thus, marker and phenotype assisted introgression studies revealed the existence of a single chromosome region which includes the high Brix QTL of green-fruited tomato fruits. The high Brix QTL (termed Brix9-2-5) was found to be associated with the centromeric portion of chromosome 9.

In order to isolate the gene or genes responsible for this phenotype further studies were conducted.

### Materials and Methods

Plant Material:

The nearly isogenic lines (NILs) for the open-field trial were planted in Akko, Israel (14-28 plants per NIL) in a completely randomized pattern. Agricultural practices and phenotypic measurements were described previously (Eshed and Zamir 1995, ibid). Glasshouse trials of the segregating recombinant families were conducted in Shekef, Israel during 1997 and 1998 in a completely randomized design.

Statistical Analysis:

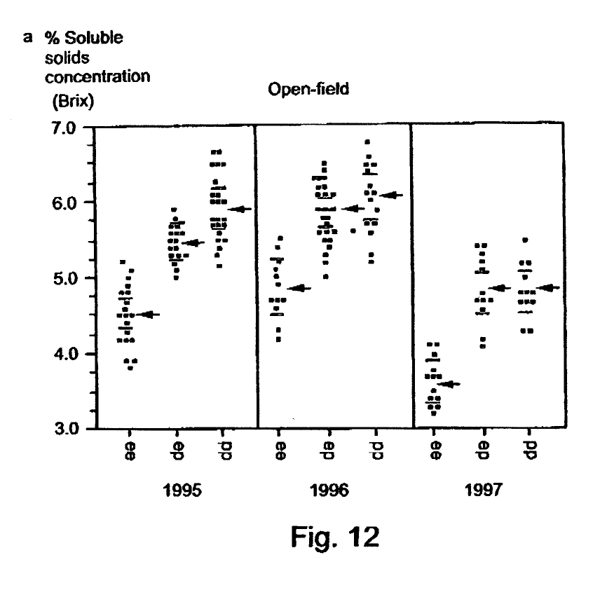

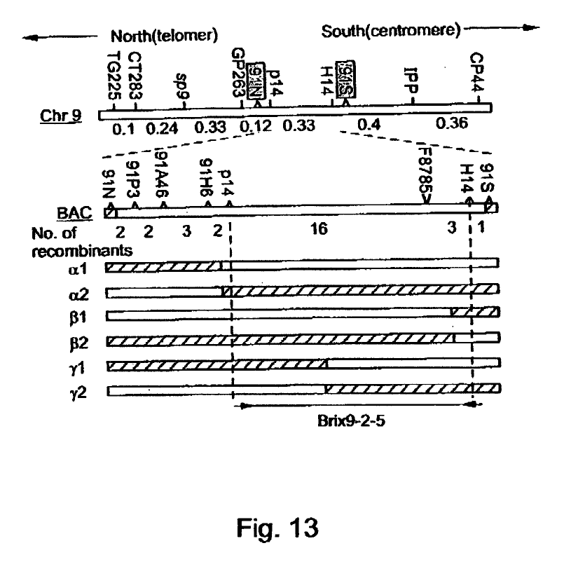

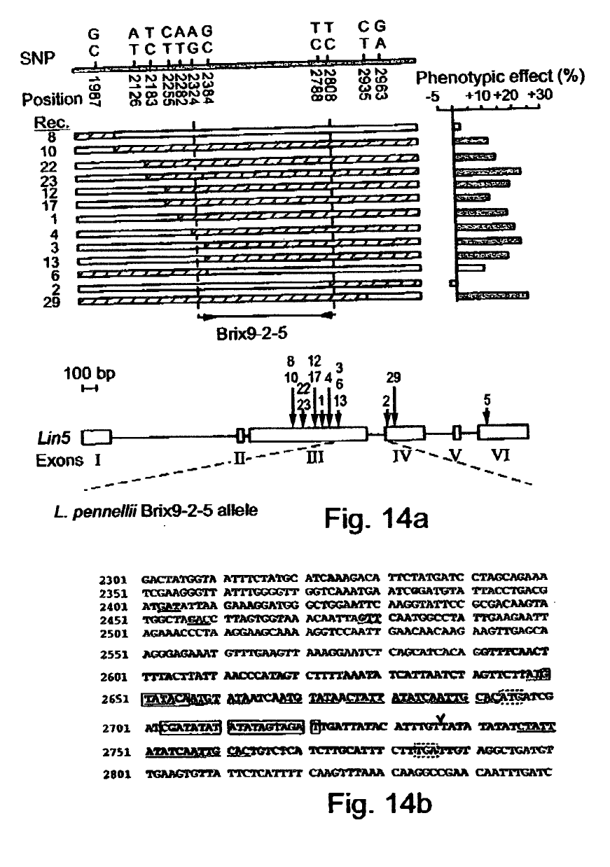

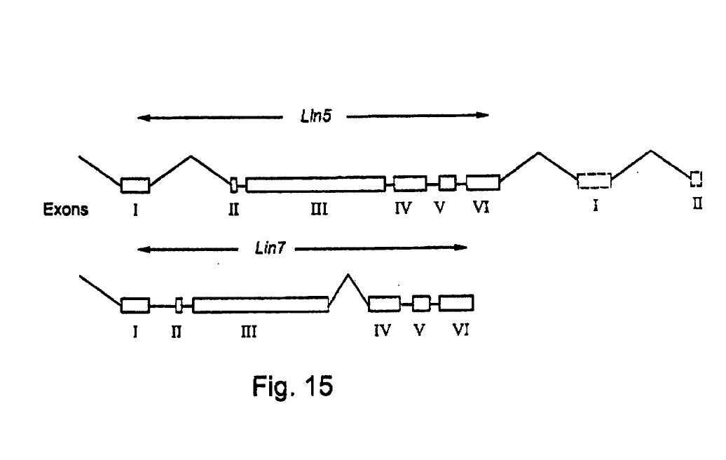

Statistical analysis was performed with the JMP V.3.1 software for Macintosh. Mean brix values were compared using the “Fit Y by X” function and “Compare with control” with an alpha level of 0.001 (Dunnet, 1955, J. Am. Stat. Assoc. 50, 1096-1121). The control phenotypic values were obtained using cv. M82 for the open-field trials (FIG. 12) and with the indeterminate line 17 for the glasshouse trials (FIG. 11). The additive effect (a), dominance deviation (d) were calculated as described above in Example 4. Mapping of Brix9-2-5 using the recombinant families was done by RFLP genotyping and a two-step analysis. In each recombinant family the brix phenotypic value for the L. pennelli homozygotes, was compared to that of line 17 and expressed as a percentage of the control (FIG. 14a). Recombinant families containing a common marker-defined L. pennelli chromosome segment were grouped and the mean phenotypic effects for the groups were calculated (FIGS. 13 and 14a).

Nucleic Acid Analysis:

The different segregating populations were subjected to RFLP analysis as previously reported (Eshed and Zamir 1996, ibid). A bacterial artificial chromosome designated BAC91A4 was isolated, subcloned, sequenced and assembled by the Sequencer software package. The nucleic acid sequence of the 13 Kb insert of BAC91A4 is presented in SEQ ID NO:1. DNA of the homozygous recombinants was used as a substrate for PCR, using the primers 5′-TTTGGGCTCATTCAGTCTCA-3′ (SEQ ID NO:2) aid 5′-AAATTGTTCGGCCTCGTT-3′ (SEQ ID NO:3) in order to amplify a 1,200 bp portion of the Lin5 gene (FIG. 14b). The PCR products were cloned and sequenced using the pGEM-T easy vector by Promega. PCR was performed using PCR Supermix (Life Technologies) with 35 cycles of 30 sec at 94° C., 30 sec at 52° C. and 1 min at 68° C., followed by 30 min at 68° C.

### Experimental Results

For fine mapping of Brix9-2-5, 7,000 F2 progenies of the NILs hybrids (described under Example 4) were subjected to RFLP analysis. Of 145 recombinants identified between the CP44 and TG225 markers (FIG. 13), 29 were further localized between the two ends of a BAC clone (BAC91A4) (FIG. 13). For each of the 29 recombinant families, 48 selfed progenies were genotyped with the appropriate segregating markers and analyzed for brix. On the basis of common introgressed segments in the 29 recombinant families, six recombination groups were generated (FIG. 13). Group α1 included seven families that contained the L. pennelli segment north of 91H6, none of which showed a significant effect on brix. The reciprocal recombination group α2 contained the L. pennellii segment south of p14 and showed a significant increase in brix. The α groups placed the QTL south of 91H6. Using the same procedure, groups β and γ located Brix9-2-5 between H14 and p14. To narrow the position of Brix9-2-5, 18-Kb spanning p14 and H14 was sequenced and used to design different primer pairs that amplified polymorphic products (in size or restriction pattern) between the parental lines. These products were genetically mapped using the 29 recombinants and one of these PCR markers (F8785, FIG. 13) co-segregated with the brix QTL. This 1 Kb genomic interval, represented by F8785, was sequenced in both the parental types and the recombinants. Based on nucleotide polymorphisms (NPs), 13 families were shown to be recombinants within this 1-Kb fragment. The phenotypic effects for each of the 13 families were used to determine the location of Brix9-2-5 on the NPs map (FIG. 14b, SEQ ID NO:4). Recombinants 3, 13 and 6 delimited the Brix9-2-5 to a region south of position 2324 (FIG. 14b) in a manner consistent with the mapping of the rest of the recombinant families to the north. Recombinant 2 delimited the QTL to the region north of position 2808 (FIG. 14b, SEQ ID NO:4), a conclusion that is in agreement with the mapping of recombinants 5 (a member of group β1; FIG. 13) and 29. This NPs mapping positioned the Brix9-2-5 between positions 2324 and 2786 of SEQ ID NO:4.

A GeneBank search revealed that the terminal portions of the brix QTL 484-bp interval contained regions encoding Lycopersicon apoplastic invertase (Lin5 GeneBank Accession number X91389) which is expressed exclusively in flowers and fruits (Godt et al, 1997 Plant Physiol. 115, 273-282) and for which a complete nucleic acid sequence is yet to be determined.

A comparison of the genomic DNA sequenced in the present study (SEQ ID NO:1) and the cDNA sequence of Lin5 resolved the genomic sequence of Lin5 (SEQ ID NO:4) which includes six exons that encode the invertase protein (SEQ ID NO:5). The 484-bp interval spans a 3′ portion of exon 3, intron 3 and the 5, portion of exon 4 (FIG. 14b). Lin5 is a member of a small family of genes encoding apoplastic invertases which irreversibly cleave sucrose into glucose and fructose. In most plant species, assimilated carbon is transported as sucrose. The extracellular invertases maintain a gradient of carbohydrates, from the source parts of the plant, to the developing sink tissues. In addition to sucrose hydrolysis, invertase plays a central role in regulating, amplifying, and integrating different signals that lead to source-sink transition (Roitscel, 1999, Curr. Opin. Plant Biol. 2, 198-206). The activity of this enzyme changes the sugar influx, and thus alters the expression of sugar-responsive genes in a manner that is yet unclear (Stunn and Tang, 1999. Trends Plant Sci. 4, 401-407).

The proposed cDNA sequence of the Lin5 invertase from L. esculentumi (SEQ ID NO:6) is identical in it's 3′ region to the partial Lin5 cDNA sequence. In addition a cDNA library from L. pennelli was screened and the full length pLin5 cDNA clone was isolated and sequenced.

The novel invertase gene associated with the brix QTL which was isolated as part of this study was designated pLin5. The proposed cDNA sequence of this gene displayed a high degree of homology (identity) (97.7%) between the L. pennelli and L. esculentum species, while homology to other isolated invertase cDNAs was less than 76% (Table 8).

Tables 5 below details the nucleotide coordinates for the various regions in the eLin5 gene (numbers refer to SEQ ID NO:1).

TABLE 5
Nucleotide coordinates of the various regions in the Lin5 Gene

| Nucleotides | Description | comments |
|---|---|---|
| 1-4849 | Lin5 promoter | |
| 4850-5048 | Lin5 exon 1 | 4850 - start codon (#1 in the eLin5 |
| | | sequence, SEQ ID NOs:4 and 6) |
| 5049-6332 | Lin5 intron 1 | |
| 6333-6341 | Lin5 exon 2 | Conserved in plants |
| 6342-6418 | Lin5 intron 2 | |
| 6419-7440 | Lin5 exon 3 | |
| 7441-7619 | Lin5 intron 3 | Include a 30 aa ORF in L. |
| 7620-7864 | Lin5 exon 4 | |
| 7865-8054 | Lin5 intron 4 | |
| 8055-8154 | Lin5 exon 5 | |
| 8155-8285 | Lin5 intron 5 | |
| 8286-8670 | Lin5 exon 6 | 8463 - stop codon |

Using blastn (Entrez) the full sequences of the L. pennelli pLin5 (SEQ ID NO:6) were compared to non-redundant (nr) and expressed sequence tag (est) libraries. Homologous sequences are presented in Table 6 and the degree of homology in Table 7.

TABLE 6
Sequences displaying homology to the L. pennellii Lin5 cDNA

| | NID | Species | PID | Blast score (gapped) |
|---|---|---|---|---|
| 1 | 3608172 | L. esculentum | 3608173 | 5e−60 |
| 2 | 313128 | S. tuberosum | 313129 | 3e−55 |
| 3 | 551258 | N. tabacum | 551259 | 6c−47 |
| 4 | 170361 | L. esculentum | 170362 | |
| 5 | 2177080 | L. esculentum | 546937 | |
| 6 | 2175258 | L. esculentum | 287474 | |
| 7 | TC4315 | L. esculentum | | |
| 8 | pLin5 | L. pennellii | | |
| 9 | eLin5 | L. esculentum | | |
| 10 | eLin7 | L. esculentum | | |

TABLE 7
Pairwise percent identity between cDNAs of the various invertases presented in Table 7

| | 3608172 | 313128 | 551258 | 170361 | 2177080 | 21775258 | TC4315 | pLin5 | eLin5 | eLin7 |
|---|---|---|---|---|---|---|---|---|---|---|
| 3608172 | | 76.0% | 82.9% | 63.4% | 63.2% | 64.2% | 61.9% | 75.6% | 75.0% | 75.8% |
| 313128 | 76.0% | | 76.4% | 62.4% | 62.3% | 63.1% | 63.6% | 74.9% | 73.9% | 74.5% |
| 551258 | 82.9% | 76.4% | | 61.6% | 61.2% | 62.1% | 62.7% | 73.8% | 73.8% | 76.5% |
| 170361 | 63.4% | 62.4% | 61.6% | | 97.7% | 98.6% | 68.1% | 65.6% | 64.4% | 62.1% |
| 2177080 | 63.2% | 62.3% | 61.2% | 97.7% | | 91.5% | 68.0% | 65.3% | 64.4% | 61.8% |
| 21775258 | 64.2% | 63.1% | 62.1% | 98.6% | 91.5% | | 68.9% | 66.5% | 65.2% | 62.8% |
| TC4315 | 61.9% | 63.6% | 62.7% | 68.1% | 68.0% | 68.9% | | 67.7% | 67.2% | 61.9% |
| pLin5 | 75.6% | 74.9% | 73.8% | 65.6% | 65.3% | 66.5% | 67.7% | | 97.7% | 79.6% |
| eLin5 | 75.0% | 73.9% | 73.8% | 64.4% | 64.4% | 65.2% | 67.2% | 97.7% | | 79.7% |
| eLin7 | 75.8% | 74.5% | 76.5% | 62.1% | 61.8% | 62.8% | 61.9% | 79.6% | 79.7% | |

TABLE 8
Pairwise percent identity between proteins translated from the cDNA
sequences of pLin 5 eLin 5 and eLin 7 and database sequences

| | 3608173 | 313129 | 551259 | 170362 | 546937 | 287474 | TC4315 | pLin5 | eLin5 | eLin7 |
|---|---|---|---|---|---|---|---|---|---|---|
| 3608173 | | 77.7% | 85.9% | 45.0% | 44.3% | 44.0% | ND | 72.5% | 72.2% | 72.9% |
| 313129 | 77.7% | | 79.8% | 43.4% | 43.1% | 39.8% | ND | 74.3% | 73.1% | 72.7% |
| 551259 | 85.9% | 79.8% | | 44.7% | 44.0% | 40.0% | ND | 74.8% | 74.3% | 76.6% |
| 170362 | 45.0% | 43.4% | 44.7% | | 99.1% | 98.0% | ND | 44.0% | 44.2% | 43.1% |
| 546937 | 44.3% | 43.1% | 44.0% | 99.1% | | 97.1% | ND | 43.5% | 43.7% | 42.4% |
| 287474 | 44.0% | 39.8% | 40.0% | 98.0% | 97.1% | | ND | 40.0% | 40.0% | 38.7% |
| TC4315 | ND | ND | ND | ND | ND | ND | | | | |
| pLin5 | 72.5% | 74.3% | 74.8% | 44.0% | 43.5% | 40.0% | ND | | 96.8% | 77.4% |
| eLin5 | 72.2% | 73.1% | 74.3% | 44.2% | 43.7% | 40%% | ND | 96.8% | | 76.7% |
| eLin7 | 72.9% | 72.7% | 76.6% | 43.1% | 42.4% | 38.7% | ND | 77.4% | 76.7% | |

The proposed translated protein of eLin5 (SEQ ID NO:6) shows a high degree of sequence identity to L. Pennelli and L. esculentum, while identity to other invertase proteins (partial sequences) for both pLin5 and eLin7 was less than 75%. The identity between pLin5 and eLin7 was about 77% (Table 8).

The homology (identity+similarity) between the apoplastic invertases (partial sequences) to the eLin5 translated protein sequence is 85-86% (not shown), whereas for the vacoular invertases the homology is below the detection on threshold of the blast search.

Comparison of the L. pennelli and L. esculentum sequences revealed several differences that may be responsible for the effect of Brix9-2-5 (FIG. 14b).

The L. pennelli third intron was longer than its corresponding sequence in L. esculentum (201 bp vs. 179 bp) and included two 18-bp perfect direct repeats, as compared to a difference of one nucleotide between the direct repeats of L. esculentum. L. pennelli carried a 7-bp triple repeat 5 to the first direct repeat, while in L. esculentum both the triple repeats were deleted. These repeats may regulate the expression of Lin5 or other genes, as was recently demonstrated for a 73-bp enhancer with similar structures in rat (Hung and Penning, 1999, Mol. Endocrin. 13, 1704-1717).

The mapping of the brix QTL was facilitated by the nearly isogenic nature of the phenotyped segregating populations where all the genetic variation for the quantitative trait was associated with the introgressed segment. The recombination hotspot created multiple isogenic chimeric alleles that delimited the QTL to a defined sequence. This hotspot, which may be associated with the direct repeats in intron 3, created 13 recombinants within a 948-bp interval as compared to only 16 recombinants for the rest of the 100-Kb BAC. This observation is consistent with studies in maize, where intragenic recombination frequencies were found to be several times greater than recombination between genes (Dooner and Martinez-Ferez, 1997, Plant Cell 9, 1633-1646 (1997).

Much of our understanding of development is based on analysis of mutants which display a loss of function. However, the variation of greatest interest is often quantitatively inherited and originates from natural populations. Tao determine the molecular basis of such traits, it is necessary to clone the genes and devise molecular and genetic complementation approaches sensitive enough to detect minor variations in gene expression pattern and function. This study highlights the potential of wild species alleles for unraveling novel variations which can be potentially useful to agricultural production.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. An isolated nucleic acid comprising a genomic, complementary or composite polynucleotide sequence encoding a polypeptide having an invertase activity in an apoplastic environment and an N terminal amino acid sequence serving for secretion into an apoplast.

2. The isolated nucleic acid of claim 1, wherein said polypeptide is at least 80% homologous to SEQ ID NO:5 as determined using the BestFit software of the Wisconsin sequence analysis package, utilizing the Smith and Waterman algorithm, where gap creation penalty equals 8 and gap extension penalty equals 2.

3. The isolated nucleic acid of claim 1, wherein said polynucleotide is hybridizable with SEQ ID NOs:1, 4 or 6 under hybridization conditions of hybridization solution containing 10% dextrane sulfate, 1 M NaCl, 1% SDS and 5×10⁶ cpm ³²p labeled probe, at 65° C., with a final wash solution of 0.1×SSC and 0.1% SDS and final wash at 60° C.

4. The isolated nucleic acid of claim 1, wherein said polynucleotide is at least 80% identical with SEQ ID NO:6 as determined using the BestFit software of the Wisconsin sequence analysis package, utilizing the Smith and Waterman algorithm, where gap weight equals 50, length weight equals 3, average match equals 10 and average mismatch equals −9.

5. The isolated nucleic acid of claim 1, wherein said polypeptide is as set forth in SEQ ID NO:5 or portions thereof.

6. The isolated nucleic acid of claim 1, wherein said polynucleotide is as set forth in SEQ ID NO:6 or portions thereof.

7. An isolated nucleic acid comprising a genomic, complementary or composite polynucleotide sequence encoding a polypeptide having an invertase activity, said polypeptide is at least 80% homologous to SEQ ID NO:5 as determined using the BestFit software of the Wisconsin sequence analysis package, utilizing the Smith and Waterman algorithm, where gap creation penalty equals 8 and gap extension penalty equals 2.

8. The isolated nucleic acid of claim 7, wherein said polynucleotide is hybridizable with SEQ ID NOs:1, 4 or 6 under hybridization conditions of hybridization solution containing 10% dextrane sulfate, 1 M NaCl, 1% SDS and 5×10⁶ cpm ³²p labeled probe, at 65° C., with a final wash solution of 0.1×SSC and 0.1% SDS and final wash at 60° C.

9. The isolated nucleic acid of claim 7, wherein said polynucleotide is at least 80% identical with SEQ ID NO:6 as determined using the BestFit software of the Wisconsin sequence analysis package, utilizing the Smith and Waterman algorithm, where gap weight equals 50, length weight equals 3, average match equals 10 and average mismatch equals −9.

10. The isolated nucleic acid of claim 7, wherein said polypeptide is as set forth in SEQ ID NO:5 or portions thereof.

11. The isolated nucleic acid of claim 7, wherein said polynucleotide is as set forth in SEQ ID NO:6 or portions thereof.

12. A nucleic acid construct comprising the isolated nucleic acid of claim 1.

13. The nucleic acid construct of claim 12, further comprising a promoter for regulating expression of the isolated nucleic acid in an orientation selected from the group consisting of sense and antisense orientation.

14. The nucleic acid construct of claim 12, further comprising a positive and a negative selection markers for selecting for homologous recombination events.

15. A plant cell, tissue or a whole plant comprising the nucleic acid construct of claim 12.

16. A nucleic acid construct comprising the isolated nucleic acid of claim 7.

17. The nucleic acid construct of claim 16, further comprising a promoter for regulating expression of the isolated nucleic acid in an orientation selected from the group consisting of sense and antisense orientation.

18. The nucleic acid construct of claim 16, further comprising a positive and a negative selection markers for selecting for homologous recombination events.

19. A plant cell, tissue or a whole plant comprising the nucleic acid construct of claim 16.

20. An isolated nucleic acid comprising a genomic, complementary or composite polynucleotide sequence hybridizable with SEQ ID NOs:1, 4 or 6 under hybridization, conditions of hybridization solution containing 10% dextrane sulfate, 1 M NaCl, 1% SDS and 5×10⁶ cpm ³²p labeled probe, at 65° C., with a final wash solution of 0.1×SSC and 0.1% SDS and final wash at 60° C.

21. An isolated nucleic acid comprising a genomic, complementary or composite polynucleotide sequence at least 80% identical with SEQ ID NOs:1, 4 or 6 as determined using the BestFit software of the Wisconsin sequence analysis package, utilizing the Smith and Waterman algorithm, where gap weight equals 50, length weight equals 3, average match equals 10 and average mismatch equals −9.

22. An isolated nucleic acid comprising a polynucleotide sequence as set forth in SEQ ID NOs:1, 4 or 6.

23. An isolated nucleic acid comprising a polynucleotide sequence encoding a polypeptide as set forth in SEQ ID NO:5.

24. A recombinant protein comprising a polypeptide having an invertase activity in an apoplastic environment and an N terminal amino acid sequence serving for secretion into an apoplast.

25. The recombinant protein of claim 24, wherein said polypeptide is at least 80% homologous to SEQ ID NO:5, as determined using the BestFit software of the Wisconsin sequence analysis package, utilizing the Smith and Waterman algorithm, where gap creation penalty equals 8 and gap extension penalty equals 2.

26. The recombinant protein of claim 24, wherein said polypeptide includes at least a portion of SEQ ID NO:5.

27. The recombinant protein of claim 24, wherein the protein is encoded by a polynucleotide hybridizable with SEQ ID NOs:1, 4 or 6 or a portion thereof under hybridization conditions of hybridization solution containing 10% dextrane sulfate, 1 M NaCl, 1% SDS and 5×10⁶ cpm ³²p labeled probe, at 65° C., with a final wash solution of 0.1×SSC and 0.1% SDS and final wash at 60° C.

28. Tile recombinant protein of claim 24, wherein the protein is encoded by a polynucleotide at least 80% identical with SEQ ID NO:6 or portions thereof as determined using the BestFit software of the Wisconsin sequence analysis package, utilizing the Smith and Waterman algorithm, where gap weight equals 50, length weight equals 3, average match equals 10 and average mismatch equals −9.

29. A recombinant protein comprising a polypeptide as set forth in SEQ ID NO:5.

30. A recombinant protein comprising a polypeptide at least 80% homologous to SEQ ID NO:5 as determined using the BestFit software of the Wisconsin sequence analysis package, utilizing the Smith and Waterman algorithm, where gap creation penalty equals 8 and gap extension penalty equals 2.

31. A method of increasing a level of a monosaccharide in a plant tissue, the method comprising the step of expressing in the plant tissue a polypeptide having invertase activity, wherein said polypeptide is at least 80% homologous to SEQ ID NO:5 as determined using the BestFit software of the Wisconsin sequence analysis package, utilizing the Smith and Waterman algorithm where gap creation penalty equals 8 and gap extension penalty equals 2.

32. A method of increasing a level of a monosaccharide in a plant tissue, the method comprising the step of expressing a polypeptide having invertase activity, wherein said polypeptide is encoded by a polynucleotide hybridizable with SEQ ID NOs:1, 4 or 6 or a portion thereof under hybridization conditions of hybridization solution containing 10% dextrane sulfate, 1 M NaCl, 1% SDS and 5×10⁶ cpm ³²p labeled probe, at 65° C., with a final wash solution of 0.1×SSC and 0.1% SDS and final wash at 60° C.

33. A method of increasing a level of a monosaccharide in a plant tissue, the method comprising the step of expressing a polypeptide having invertase activity, wherein said polypeptide is encoded by a polynucleotide at least 80% identical with SEQ ID NO:6 as determined using the BestFit software of the Wisconsin sequence analysis package, utilizing the Smith and Waterman algorithm, where gap weight equals 50, length weight equals 3, average match equals 10 and average mismatch equals −9.


SEQUENCE LISTING (9 sequences)

SEQ ID NO: 1 - DNA, 13431 bp, Lycopersicon pennellii
  misc_feature (1)..(13431): Bacterial artificial chromosome - BAC91A4 13kB insert
gatccaacgcgggtacaacaatatttttggagagtttgagcaacataggatgagagggggatgaagaaagtggactaaacagactcacatttctcatcgaaagtacagtgaaaagaatcatatcgaacacacccctaaccaatcccttctttcgtagatcatgtgatctgagcctgcctgcaacgagccacttgccgaggcaagtgaagttggcgagccgtatgatggacaactctatcctgcagtttggtaattgtaattcaaaaaaataattccaagagataaaaaatcaattcttgtttgagaaaactatgtgctgaattggacaagtttgggggccaatgaagtcaatctactaatttccgaaaaatcaatggactaacaaacacgaaaaaatacctaaaagtacttactacatgaggccaaaacccatcaagtactacatgaacaactaattaaacaccaatttcacatttttctcgttaaaatttttatctttcttaacttactaagtttctaaggtagaacattcaataaatcgaggtattttaaaactcaatgtctaaattctgaatctgtcgctgtcgatatatttctccaattcattgaactaaggaatattttactttatgggaaattaagtgaaagctgcaaaagctaaatcttataattagtatagttcaccacatacattatgccttcttattgtaaccaattaatttttgtggccactacaagaattaagacaaacatcttttcaattataagacaaattgcctaatccaactatggcaaaagaacaacttgctcacacacatacacacactaaagaaatatacgattcatcgatatcctacgagtcgttatatggtgtctgctgttgataagaaaacgaaccttaagcctaactcaatttgtagtttttatgggtatttgaattcaacgagatatgttgcactttttttttttgctgaataatgaaagggaataaaatcttgggcaaattacataactcacatacttttaagttaaaattacaatttatctcttagaagtttgtatttacaaaaatctttcaaacggatacattaatggagggcccagatacattaatagccaaaaaatcaaagcactgagatattaaaaagagggccgaatacgtgcattgatatattaaaaaaagggtcgaatacattaatagagtgtcggatacattaatgcagggtttggatacattaatggccaaaagatcaaagcactgatacattaaatagatgatcatacattaaatagatgatcagatacatgcgctgatacattaaaaagaggatgaatacattaatggagtgtctgatacattaatatttttttttgactttagagaaaaatgaggaattttagaaatttgtaaaacttataaggggataatgataataagtaaaataaaagatatgaaatttttgtaattattcctaaaattttctttttgttattgttaatatatatatatataaaggttgagcttttttttttctctcaagaatatgattattacatcatgttgacatgggaactaaaaatttatttattatttaaaaattaggtatttatggataagggaaattagtgcaatataaaagtttgatatagaagttacaacaattttagaggtgcacatgtattaccaaatttccaataccaaatgcatatgttacacttaggttctttttgaggtatcaccaaattgtgttaaaattgtacttagaacttttccaaagttattgtgaaaacttgtcatcaagtgagcaaaaaccttagtggaagtgcatgtaggaagcttatcatcaagatttcttgtgtttttgatgaaagaatgcttaggtaggggcggattcagaatctgaatatatcattatagatttaaattcgagattcttccattcatttcgtttatagggtttacgtttgaggtatacatactatttgtatttatttagtaaattctttaaaggcttaagtcatacatactattttcttccccttctttcaccacccatcaccgacatcacaaatatttaaatatattgtggtaagaaatagaattaaaaataattttgaggaaactccatttttttcgacgaatctgaattattatttttttaaatcgttaagaaatttaaatcctaaaaaactcacaacacagaagaaaaaaagtatcaataaatttaaattaatatttaagaaagtagtgatattgggagaaataccaattacaataatgagtaagattcaaggagaaagataggtggatggattgaaaaacaaattcatataattttgaattgaaaggaaaaagagtcaaattcaaaattactatcgaaatacttcgattttgtgtctcgtgctaagggaaatggtgaacgaatgtcagaagagggatggggtgggatgttctgggaagaagaagaagaaaaggggaacgaaatatttttttttaattattttttttagtgaaaacataatttttgtttattttatttttttaatttatgttttaagtgtgtttatgggcccaatgccacatgtctcttttttattagctattatttcacgttagcagcgagtgtaggacattctctctttacttttaattaattgtcaaaatatatatatatctctttaaaatacactattaaataatacatcgataaaatatcttttccaaaatttgattattattacaataatttcgatcaaaattcatgaggtgttttttctttttttatatacaattcactaatatttggcaatggaatccactaagtaaatcaaaggtggaaaaactattaactgcaaatgttgcaagacatatttatgcaaatgaaaagtagaaagctaatactttttgccaaattcatcaaaaaaatgtaatgtgtaaaatgcactttattgttgttatgcatatatgttcatcacctattcatctatccattatatttcattttcaataattactgtggtcctattcattttattttggaactcaaatatcatgattacatattatttaaaattcgcgttacgatgaaatataggcgaaaaaaactattaatctttctcatgaggtgaggtgaggcgagaaaaacaaaacgttatatattatttgatgtcttgtgtgaggtgtcactattctagttgtgttttatctccgtctatactgaagtttaaaagtacatatatagctaatcgaaaacatatttttatcattgaaaattacaaattttttatgtacgtctttgttgaaacgtcgataaaaatataaatattaatcggaagtcggtcaaaaatatttctgacaaaacaattctattccatcgataatttttactcttttttttttttaagtattgatcaatttattcatctagaaggataaagtctacatttttatttgttttaaacatatatttacacaaatggtcttttcaactatatgttatagaatttgtgaattaaatcaaatattatttttgttatgattttaggttcgagtcgcttataaaaagattgtagttcatagccaaaattcttctcaaaggcggaataataattttaaatttatgagcgttttgaattctaaaacagataatttatttaataaattatattatacatatgagagataatttattcaataaattatttatttatattatataagtggaactttaatataaatatacgaaacgcttatgctaaaattgataacttgtatttattttaattttaatgtttttggagggagaaggtaggtaaaatttgaacatatattggtacgtgacaattctaaaatgtcttgaaaggtcgtgtcggtgctacgatttttaacgagagagtttaaaatttaaagaagtaaaatagataatttaattatgtataaataataatattttctatcagagtggatccgaacgaatattattttgaatattattttgaattaaggcaaaacaaacactgtatatatgattaattttcgtcaaccaatagtcccttctctagttttctatacgtcaaattagattgtttaaacacaaatacttatatgataatcttttacgtgtaatcgatacatagaaaataacgttattattttttaccaagagtcttatatatactttatttgtaaattattacgtcaggctttatacgttaaattattttgtcatgtaaattaactttactttagtaatatgaaaagtctatacaagcatcgagtgtataatttgacttttattcgattaaaaaaatacaccttatatatacaaattaagattatattttatatttgtatagtaaatattgacctatttattgacgatataaaatcaccccctgatcgtcatgcttgaatgaagacacctgattgatttgattttttcacatgacccctcctcgctcttctcttttacgagttggaattttcatatagttttaattatcatgataaaaatgagggaatatattttttaaaattatatattgatacatatatcgagataaaaattaaaactaaatctcataaaaatgaaaattttgatttcgtcactaaacgaaagaaagagaagtcacgcgtaagaaaaatacggctaatataattgaagtttatcttttaagaaacattatttacatccattttattttaattaaagaaaaagaaaatataaaaatcaaaccgttttttttatccactcatcatagttggtttttgtctataaataatccaatattctttcttttcttcttcattattcttcaatatctttctccaaaatcacaaaaaaaaaaaatggaattatttatgaaaaactcttctctttggggtttaaaattttatttattttgcttatttataattttatcaaacattaatagggcatttgcttctcataatatttttttggacttgcaatcttcaagtgctattagtgtcaagaatgttcatagaactcgttttcattttcaacctcctaaacattggattaatggtatgttcattttttttttattttatataacatgcgataaatttaacgttagcaatgtggtttgttatttaaattcgaatttgattatatgactttgcttatataaatatacatagtaataaaagtttgtgtataaatgcatgtcatatacatttattgacttggtatatatatcagtacgattaaattaattgatggtgcaattaatattgcattatttaggtgataaaactacagaaattaacgaaaatattttttttatatagagaagttcaaatgttgagggttcttttatggttacattggtttaaaatgttttttgttaactatctttatagctacatatatataagagtgatcattctttatatttcaaaattatatctacatacacacatatacatcattatgtggttcatttatggtagttttcagtattcgatatttatttttaagtttaatttatttaaatctgcgttaaaatatctcactttgaaagatagaatcactcctgaccaactatgagtaactcgattctcaaaatttaaattcggaattagattaattatcatggcaagagaactaccacgttttggataagaatgtgcaaaagagagaaagaaacatgaaatatataaaaacctaagattttggccatggaaagttaggtgcgaattaatttgttgaaggcaccctttattattattattataattattattattattaatgaaatatagtgacatttcatactcatatattgtgtgcatttaattaatatatgtaggtcttatgttaatttaaacttaccaaacatattgtctcttataaagttgactccccccctcaaccgccaaccccacccccacccccaccccacccaaaaaaaatacctcatcaatttcggtttttatatgactcaattttcttgtttaatttgttatctacagaacggactactttctatatcattctacataatatgtatattttttataatccaataaatctcatgacacgttttcagatcataattttgcaaacacctttttctttattttttaattaggtatatcacataaattaaaaggattcattaattttcgcagagaaaactaattagtttctgtgtttttcacctttcatttattaattactacataatttttaatcaataattgatgaaagactatgtaatgtattctattatcttcactaatcattttttttttgtataattcttatatggtctctctccattggatgcctttcaaatatacaaagaccctaatggtaagttagattatttttcatttaattttatcaataactcaatgatattattgattttcattttatttttcaaacagcaccaatgtattataatggagtgtatcatttattctatcaatacaatccaaaaggatcagtatggggcaatattatttgggctcattcagtctcaaaagacttgataaattggatccatttagaacctgcaatttatccatccaaaaaatttgacaagtatggtacttggtctggatcatcaactattttacctaataacaaacctgttatcatatacaccggagtagtagattcgtataataatcaagtccagaactacgccatcccggctaacctatctgatccatttcttcgtaaatggatcaaacctaacaacaacccgttgatcgtccctgataacagtatcaatagaactgagtttcgcgatccaactacagcttggatgggccaagatgggctttggaggattttaatagcaagtatgagaaaacatagagggatggcattgttgtatagaagtagagattttatgaaatggatcaaagcccaacatccacttcattcatctactaatactggaaattgggagtgtcctgattttttccctgtattatttaatagtaccaatggtttagatgtatcgtatcgcggaaaaaatgttaaatatgtcctcaagaatagtcttgatgttgctaggtttgattattacactattggcatgtatcacaccaaaatagataggtatattccgaataacaattcaattgatggttggaagggattgagaatcgactatggtaatttctatgcatcgaagacattctatgatcctagcagaaatcgaagggttatttggggttggtcaaatgaatccgatgtattacctgacgatgaaattaagaaaggatgggctggaattcaaggtattccgcgacaagtatggctaaaccttagtggtaaacaattacttcaatggcctattgaagaattagaaaccctaaggaagcaaaaggtccaattgaacaacaagaagttgagcaagggagaaatgtttgaagttaaagggatctcagcatcacaggtttcaacttttccttattaaactatagtcttttaaatatcattaatctacttcttatatgtataatcaatgtataactattatatcaaatgcacatgatcgattgattatacatttgctatatatatatctctattatatcaattgcactgtctcatcttgcatttctttgatcgtaggctgatgttgaagtgctgttctcattttcaagtttgaacgaggccgaacaatttgatcctagatgggctgacctatatgcccaagacgtttgtgccattaagggttcgactatccaaggtgggcttggaccatttgggcttgtgacattagcttctaaaaacttagaagaatacacacctgttttcttccgagtgttcaaggctcaaaaaagttataagattctcatgtgctcagatgctagaaggtttgtttcttcaatccaattaattgtaatgatcgaagttcacatcttctccaaattgagtaaatcgagaattataatgacccgactttgatatcatgataagaaatgcatttacttatagatcgcccgttagtgtcattaaaaaactctaaccttgtttaggttttttttttttttaattaatgagcagatcttccatgagacaaaatgaagcaatgtacaagccctcatttgctggatatgtagatgtagatttagaagacatgaagaagttatctcttaggagtttggtaagttttgctttcacaatttttatttatttataatttatttgatcaaaactttcaagattcgattaatttgaagagtaacgatttgtgtttgactaatcaatttgtatcatatgcatatttttttttagattgataactcagtagtggaaagtttcggtgctggtggcaaaacatgcataacatcaagggtgtatccaactttagcgatttatgataatgcacatttatttgtttttaacaatggctctgagacaatcacaattgagactctgaatgcttggagcatggatgcatgtaagatgaactaaatattttcaaaaaaattggaattatgtctacaattatatatgtctaaagagacaaaaattgtgttaaatttaacagtagatgatgttcacaaaaatcctctataattgtctctaatttattttggtgaatttagaaggcaaagtgtgtgtatggatttttctagtaccatatatatatatattaagtaagaaatttgttagctttcctttttgttttgtaacataatcaaatgtgtggtcttatgtagaactaatatttggtaatattaggcaagttgttatgtgacttattttattcaaaaatataataagaagttcaaagagaagagtacaagtaagtaagtaagcagagacgaatcctggatttaaagggtctggctatattaatgtttttttaatttaagcattagcgattcgccttgcaagtaatcgataggacaaaagttttaccttactaattctattgaggcaccaaatccctatgaaaaagcatgtaaaatatgagaagacgaaagaattaaataggttataattattgtataatttataacacactttatgataatattacaaataagaatatcgaatatttaattaatgacgaactataaaagcaaagaaggaaggatgagcttccaaaaacaatcgcaaatgaataaagatgcccaaaatagagtaacctaacgaagtcgatacttccattcataatcaaatctgttcaaaaacacttgatggtttatttttaactttaagagatgtatcatatcgtctcttattattcttttagggctattcgccgtaggaataaaatttatatgatcaaatttcacgttatataaataatgtgaagaaaaaacttatacttttcaaggtaacaagaaatcatgttttttttacgccttcgtggagactacttcctcgtaacaaaaaattaacattttaagtggcgactctaaaaactcgtggccagtatattagtcgccattaaacattatttttaatcatgagttcttttcttttttaatctttttttaaggtcaaatttaccactttatcttatttatttaaattgaaaaatcccaaattttgcattatttttttgaattccttttttttttacacactcaaaaagtcaaaacattaaaaaaacgaaatagcaaattaaatggcaaaagacttgttctaacaaaaaaaaaatagtaaaacagactcataaaaggtaacaataaccaacaaatcacacaaaattgtagataaatattatgcaaacaaataaaaattaataatccaatccatttatttatttttttaaaaaaaacctaaattaactctccatctttcaatcaaaaacaaactctacccatttttttcactataaatactcttcataattttcatttgttcttcattcccatgtttcttttctccttatccaaaaaaaaaaaaattaaaaaaaattatttagattaaatatcactatctgtcaaagcccaatcattaaaataaaataaaaattatggattattcatctaataaaagttctcgttgggctttgccagttatcttagtttgcttttttgtaattttattatccaataatgttgtttttgcttctcataaagtttttattcacttgcaatctcaaaatgctgtaaatgttcatactgttcatcgaactggttatcattttcagcccgaaaaacattggatcaatggtatgtttattcctttttttcgtcttttttttatatatatatataataaaacgaacatgttgtgtttagtctagatttaatactagtgatttttttgacgctaacaaataatcgagtactcaccatttgtcaatagatacattgacatgtattagtatgattttcgtcttttttcgttgtttctaatattatttaatcttcactaattttttttatttttctttgaatgatgtctcttggtcaaaacatacaatagatcccaatggtaagttaactatatttttgtatattttttaaatttattttattcttattatataatatagggaaaaaaggataaatatatccccgaactattataaatagtatgcaccagtatcctctgttatactttagagacatttttgccgtcaaaaaactagaacacatatatcctttatttatcccgatatcgaatcgattgtaccacgagtgaagggtatagctctagtttttggacggtagggcacctaaagtatgacgaagaatatctgcaaaccatttacaatagttttggatatatttgtgaactaatgatgtttgaattcttttttcatagcaccaatgtatttcaatggagtgtatcatctattctaccagtacaacccaaatggttcagtatggggtaacattgtttgggctcattccgtttcaaaagacttgatcaattggatcaatttagaacctgcaatttacccatcaaagccatttgatcaattcggtacctggtctggatcagcaaccatcctacctggtaacaagccagtcatcttgtacaccggaatcatagatgccaaccaaacccaagtccaaaactacgcaatcccagctaacttatccgatccatatctccgcgaatggatcaagccagacaacaacccattaattatagccgatgaaagtatcaacaagaccaagtttcgtgacccaacaacagcatggatgggtaaagacgggcattggagaatcgtcatgggaagtttgaggaaacacagcaggggcttagctataatgtataggagcaaagactttatgaaatgggtcaaggctaaacacccacttcactcaactaacggcactggaaactgggaatgccctgatttttacccagtttcatcgaaaggtactgatgggttggatcaatacggtgaggaacacaagtacgtgctgaagaacagtatggatcttactcgatttgagtattatacacttggaaaatacgatacgaaaaaagataggtacgttccagatccagattctgtcgatagtttgaagggattgagactcgattacggtaacttctacgcatcgaagtcattctacgatccaagcaaaaatcgaagggttatctggggttggtctaatgaatcagatatattcccagaggatgataatgcgaagggatgggctgggattcaattgattcctcgtaaagtatggcttgatccaagtggtaagcagttggttcaatggcctgtggaggaactagaaaccctaagaactcaaaaggttcaattgagcaacaagaagatgaacaatggggagaagattgaagttacaggaatcacaccagcacaggtatatatatagacttttttatttttaatttattattattattattattactctctccgttttcaaaaaaaaaatatccctattttcttttatagtctctttaatttaaaaagaatgatctattttctttttggataaccttttaactttgatttttcacgtgaaatgtttaaaatcacgagattaaagagcattttggttacatttgacataactgaaatttagaaacacaagattaaaggacattttggtacatttgacataacttgaatttaaaaccacataattaaagggcattttggtacatttgaattagaacattttgatacatttgacataacatgaatttagaaccacaagattaaaaaatcttctttcttttttcttaaatttcgttccaagtcaaattaggtcattcttttttaattactccctccgtctaattttatgtaacaacatttgaccggacggagagttttaagaaataaataaaacactttgagatgtgtaccaaattgctctccaaaaatactcacttttctctctcctcataaatgtatttgagtactatttttaaaattaagcgagtccaacaagaataaaatagaaactgtacttttaaatatttaccatataaaaaaatgtgattttttttttttgaaaactgatcaaaaagaaaatgatatcactcgacgatgaaagtgtttaataatgaaaaaacatgacaggctgatgttgaagtgacattctcatttgcaagtttggataaggcagagtcatttgatcctaaatggaatgatatgtatgcacaagatgtttgtggactcaagggtgcagatgttcaaggtgggcttgggccatttggtcttgctacattagctactgaaaacttggaagaaaacacaccggttttcttccgagttttcaaagcacagcaaaactacaaggttctcttgtgttctgacgctaaaaggtactacttattgaatttttaacttgttggtaacgttttcgacggtataatatcgagaagttgagaaattgacaaatcttttgttttatgtctgataggtcaactcttaagttcaatgaaacaatgtacaaagcttcatttgctggatttgttgatgttgatttggctgacaagaaattgtcactcagaagcttggtaacttctctttctatcgttaatcaaaaatctaaacgaacatttgaatctaaactattgaaattctttttgtagattgataattcagttatagaaacttttggtgctggtggaaagacatgtataacatcgagggtttatccaacattggcaattaacgacgaggcacatttattcgcgtttaacaacggaacggagccaatcacaattgagagtttggatgcatggagtatgggcaaagctaagatacaatattgaagaaaaaaaaaagaataaggagaaaggaactatggtaaaagggggaaatatgagattttgatggacatataagagtttgttattttgatatttcaactctattttttttgtttatcaaattagatatccctcgagtcaaaaaaaatgtatatagatttatcctctattttcaatttatgacaataatgctatgtttttgctttcaaaa

SEQ ID NO: 2 - DNA, 20 bp, Artificial sequence
  Single strand DNA oligonucleotide
tttgggctcattcagtctca

SEQ ID NO: 3 - DNA, 18 bp, Artificial sequence
  Single strand DNA oligonucleotide
aaattgttcggcctcgtt

SEQ ID NO: 4 - DNA, 3616 bp, Lycopersicon pennellii
atggaattatttatgaaaaactcttctctttggggtttaaaattttatttattttgcttatttataattttatcaaacattaatagggcatttgcttctcataatatttttttggacttgcaatcttcaagtgctattagtgtcaagaatgttcatagaactcgttttcattttcaacctcctaaacattggattaatggtatgttcattttttttttattttatataacatgcgataaatttaacgttagcaatgtggtttgttatttaaattcgaatttgattatatgactttgcttatataaatatacatagtaataaaagtttgtgtataaatgcatgtcatatacatttattgacttggtatatatatcagtacgattaaattaattgatggtgcaattaatattgcattatttaggtgataaaactacagaaattaacgaaaatattttttttatatagagaagttcaaatgttgagggttcttttatggttacattggtttaaaatgttttttgttaactatctttatagctacatatatataagagtgatcattctttatatttcaaaattatatctacatacacacatatacatcattatgtggttcatttatggtagttttcagtattcgatatttatttttaagtttaatttatttaaatctgcgttaaaatatctcactttgaaagatagaatcactcctgaccaactatgagtaactcgattctcaaaatttaaattcggaattagattaattatcatggcaagagaactaccacgttttggataagaatgtgcaaaagagagaaagaaacatgaaatatataaaaacctaagattttggccatggaaagttaggtgcgaattaatttgttgaaggcaccctttattattattattataattattattattattaatgaaatatagtgacatttcatactcatatattgtgtgcatttaattaatatatgtaggtcttatgttaatttaaacttaccaaacatattgtctcttataaagttgactccccccctcaaccgccaaccccacccccacccccaccccacccaaaaaaaatacctcatcaatttcggtttttatatgactcaattttcttgtttaatttgttatctacagaacggactactttctatatcattctacataatatgtatattttttataatccaataaatctcatgacacgttttcagatcataattttgcaaacacctttttctttattttttaattaggtatatcacataaattaaaaggattcattaattttcgcagagaaaactaattagtttctgtgtttttcacctttcatttattaattactacataatttttaatcaataattgatgaaagactatgtaatgtattctattatcttcactaatcattttttttttgtataattcttatatggtctctctccattggatgcctttcaaatatacaaagaccctaatggtaagttagattatttttcatttaattttatcaataactcaatgatattattgattttcattttatttttcaaacagcaccaatgtattataatggagtgtatcatttattctatcaatacaatccaaaaggatcagtatggggcaatattatttgggctcattcagtctcaaaagacttgataaattggatccatttagaacctgcaatttatccatccaaaaaatttgacaagtatggtacttggtctggatcatcaactattttacctaataacaaacctgttatcatatacaccggagtagtagattcgtataataatcaagtccagaactacgccatcccggctaacctatctgatccatttcttcgtaaatggatcaaacctaacaacaacccgttgatcgtccctgataacagtatcaatagaactgagtttcgcgatccaactacagcttggatgggccaagatgggctttggaggattttaatagcaagtatgagaaaacatagagggatggcattgttgtatagaagtagagattttatgaaatggatcaaagcccaacatccacttcattcatctactaatactggaaattgggagtgtcctgattttttccctgtattatttaatagtaccaatggtttagatgtatcgtatcgcggaaaaaatgttaaatatgtcctcaagaatagtcttgatgttgctaggtttgattattacactattggcatgtatcacaccaaaatagataggtatattccgaataacaattcaattgatggttggaagggattgagaatcgactatggtaatttctatgcatcgaagacattctatgatcctagcagaaatcgaagggttatttggggttggtcaaatgaatccgatgtattacctgacgatgaaattaagaaaggatgggctggaattcaaggtattccgcgacaagtatggctaaaccttagtggtaaacaattacttcaatggcctattgaagaattagaaaccctaaggaagcaaaaggtccaattgaacaacaagaagttgagcaagggagaaatgtttgaagttaaagggatctcagcatcacaggtttcaacttttccttattaaactatagtcttttaaatatcattaatctacttcttatatgtataatcaatgtataactattatatcaaatgcacatgatcgattgattatacatttgctatatatatatctctattatatcaattgcactgtctcatcttgcatttctttgatcgtaggctgatgttgaagtgctgttctcattttcaagtttgaacgaggccgaacaatttgatcctagatgggctgacctatatgcccaagacgtttgtgccattaagggttcgactatccaaggtgggcttggaccatttgggcttgtgacattagcttctaaaaacttagaagaatacacacctgttttcttccgagtgttcaaggctcaaaaaagttataagattctcatgtgctcagatgctagaaggtttgtttcttcaatccaattaattgtaatgatcgaagttcacatcttctccaaattgagtaaatcgagaattataatgacccgactttgatatcatgataagaaatgcatttacttatagatcgcccgttagtgtcattaaaaaactctaaccttgtttaggttttttttttttttaattaatgagcagatcttccatgagacaaaatgaagcaatgtacaagccctcatttgctggatatgtagatgtagatttagaagacatgaagaagttatctcttaggagtttggtaagttttgctttcacaatttttatttatttataatttatttgatcaaaactttcaagattcgattaatttgaagagtaacgatttgtgtttgactaatcaatttgtatcatatgcatatttttttttagattgataactcagtagtggaaagtttcggtgctggtggcaaaacatgcataacatcaagggtgtatccaactttagcgatttatgataatgcacatttatttgtttttaacaatggctctgagacaatcacaattgagactctgaatgcttggagcatggatgcatgtaagatgaactaa

SEQ ID NO: 5 - protein, 584 aa, Lycopersicon pennellii
MELFMKNSSLWGLKFYLFCLFIILSNINRAFASHNIFLDLQSSSAISVKNVHRTRFHFQPPKHWINDPNAPMYYNGVYHLFYQYNPKGSVWGNIIWAHSVSKDLINWIHLEPAIYPSKKFDKYGTWSGSSTILPNNKPVIIYTGVVDSYNNQVQNYAIPANLSDPFLRKWIKPNNNPLIVPDNSINRTEFRDPTTAWMGQDGLWRILIASMRKHRGMALLYRSRDFMKWIKAQHPLHSSTNTGNWECPDFFPVLFNSTNGLDVSYRGKNVKYVLKNSLDVARFDYYTIGMYHTKIDRYIPNNNSIDGWKGLRIDYGNFYASKTFYDPSRNRRVIWGWSNESDVLPDDEIKKGWAGIQGIPRQVWLNLSGKQLLQWPIEELETLRKQKVQLNNKKLSKGEMFEVKGISASQADVEVLFSFSSLNEAEQFDPRWADLYAQDVCAIKGSTIQGGLGPFGLVTLASKNLEEYTPVFFRVFKAQKSYKILMCSDARRSSMRQNEAMYKPSFAGYVDVDLEDMKKLSLRSLIDNSVVESFGAGGKTCITSRVYPTLAIYDNAHLFVFNNGSETITIETLNAWSMDACKMN

SEQ ID NO: 6 - DNA, 1960 bp, Lycopersicon esculentum
atggaattatttatgaaaaactcttctctttggggtttaaaattttatttattttgcttatttataattttatcaaacattaatagggcatttgcttctcataatatttttttggacttgcaatcttcaagtgctattagtgtcaagaatgttcatagaactcgttttcattttcaacctcctaaacattggattaatgaccctaatgcaccaatgtattataatggagtgtatcatttattctatcaatacaatccaaaaggatcagtatggggcaatattatttgggctcattcagtctcaaaagacttgataaattggatccatttagaacctgcaatttatccatccaaaaaatttgacaagtatggtacttggtctggatcatcaactattttacctaataacaaacctgttatcatatacaccggagtagtagattcgtataataatcaagtccagaactacgccatcccggctaacctatctgatccatttcttcgtaaatggatcaaacctaacaacaacccgttgatcgtccctgataacagtatcaatagaactgagtttcgcgatccaactacagcttggatgggccaagatgggctttggaggattttaatagcaagtatgagaaaacatagagggatggcattgttgtatagaagtagagattttatgaaatggatcaaagcccaacatccacttcattcatctactaatactggaaattgggagtgtcctgattttttccctgtattatttaatagtaccaatggtttagatgtatcgtatcgcggaaaaaatgttaaatatgtcctcaagaatagtcttgatgttgctaggtttgattattacactattggcatgtatcacaccaaaatagataggtatattccgaataacaattcaattgatggttggaagggattgagaatcgactatggtaatttctatgcatcgaagacattctatgatcctagcagaaatcgaagggttatttggggttggtcaaatgaatccgatgtattacctgacgatgaaattaagaaaggatgggctggaattcaaggtattccgcgacaagtatggctaaaccttagtggtaaacaattacttcaatggcctattgaagaattagaaaccctaaggaagcaaaaggtccaattgaacaacaagaagttgagcaagggagaaatgtttgaagttaaagggatctcagcatcacaggctgatgttgaagtgctgttctcattttcaagtttgaacgaggccgaacaatttgatcctagatgggctgacctatatgcccaagacgtttgtgccattaagggttcgactatccaaggtgggcttggaccatttgggcttgtgacattagcttctaaaaacttagaagaatacacacctgttttcttccgagtgttcaaggctcaaaaaagttataagattctcatgtgctcagatgctagaagatcttccatgagacaaaatgaagcaatgtacaagccctcatttgctggatatgtagatgtagatttagaagacatgaagaagttatctcttaggagtttgattgataactcagtagtggaaagtttcggtgctggtggcaaaacatgcataacatcaagggtgtatccaactttagcgatttatgataatgcacatttatttgtttttaacaatggctctgagacaatcacaattgagactctgaatgcttggagcatggatgcatgtaagatgaactaaatattttcaaaaaaattggaattatgtctacaattatatatgtctaaagagacaaaaattgtgttaaatttaacagtagatgatgttcacaaaaatcctctataattgtctctaatttattttggtgaatttagaaggcaaagtgtgtgtatggatttttctagtaccatatatatatatattaagtaagaaatttgttagcttt

SEQ ID NO: 7 - protein, 5 aa, Artificial sequence
  Beta-fructosidase motif
NDPNG

SEQ ID NO: 8 - protein, 6 aa, Artificial sequence
  Invertase catalytic motif
WECPDF

SEQ ID NO: 9 - DNA, 500 bp, Artificial sequence
  Sp9 marker probe
ttctgcaagaagataaaataatataattttttctagtaatttaaatattatatgtgaatattgtaagttaaacatgaagttcacaaggagagtatatgattatatgattaattaaagatttagacaaaattaaagggtatttttggtacgacgtaaaaataactttttagaaaatatttttgcgagtatattatttattaatgttttatactaatatagaagtcgttatttttagggaaaaaaagttcttttcaaaatatgaaataaatttctagcctagggacgaaagtcttttttttttttataactatagtaaacgtaaaatcacgtaattaaaacatttataataataaaagataaaagatctatattggttttaccaattagtacatattaggttttagtcacgttaatatgtttacttttttgttctaatattagtaattatctattaatcttgtaatagctaatttttttattttttttttgtaattgattaa